SEQUENCE LISTING (2 sequences)

SEQ ID NO: 1 - DNA, 4398 bp, Homo sapiens
  CDS (276)...(2714)
  misc_feature (1)...(4398): n = A,T,C or G
cncgcgtccgcggacgcgtgggggcgagggccgctggggccgcgaagtggggcggccgggtgggctacgagccgggtctgggctgaggggcgcggcttcgcggtggaccccagcccggcaacgggaaggcgagctctcctccaccgtccaaagtaaactttgccgctccttccgcggcgctcccgagtcctcgccgccgccgggccgccgcagtccgcgaagagccgtcctgcgtcagggcctccttccctgccccggcgcggggccactgcgccatggacgccacagcactggagcgggacgctgtgcagttcgcccgtctggcggttcagcgcgaccacgaaggccgctactccgaggcggtgttttattacaaggaagctgcacaagccttaatttatgctgagatggcaggatcaagcctagaaaatattcaagaaaaaataactgagtatctggaaagagttcaagctctacattcagcagttcagtcaaagagtgctgatcctttgaagtcaaaacatcagttggacttagagcgtgctcatttccttgttacacaagcttttgatgaagatgaaaaagagaatgttgaagatgctatagaattgtacacagaagctgtggatctctgtctgaaaacatcttatgaaactgctgataaagtcctgcaaaataaactgaaacagttggctcgacaggcactagacagagcagaagcgctgagtgagcctttgaccaagccagttggcaaaatcagttcaacaagtgttaagccaaagccacctccagtgagagcacattttccactgggcgctaatcccttccttgaaagacctcagtcatttataagtcctcagtcatgtgatgcacaaggacagagatacacagcagaagaaatagaagtactcaggacaacatcaaaaataaatggtatagaatatgttcctttcatgaatgttgacctgagagaacgttttgcctatccaatgcctttctgtgatagatggggcaagctaccattatcacctaaacaaaaaactacattttccaagtgggtacgaccagaagacctcaccaacaatcctacaatgatatatactgtgtccagttttagcataaagcagacaatagtatcggattgctcctttgtggcatcactggccatcagtgcagcttatgaaagacgttttaataagaagttaattaccggcataatttaccctcaaaacaaggatggtgaaccagaatacaatccatgtgggaagtatatggtaaaacttcacctcaatggtgtcccaagaaaggtgataattgatgaccagttacctgttgatcacaagggagaattgctctgttcttattccaacaacaaaagtgaattatgggtttctctcatagaaaaagcatacatgaaagtcatgggaggatatgattttccaggatccaactccaatattgatcttcatgcactgactggctggataccagaaagaattgctatgcattcagatagccaaactttcagtaaggataattctttcagaatgctttatcaaagatttcacaaaggagatgtcctcatcactgcgtcaactggaatgatgacagaagctgaaggagagaagtggggtctggttcccacacacgcatatgctgttttggatattagagagttcaaggggctgcgatttatccagttgaaaaatccttggagtcatttacgttggaaaggaagatacagtgaaaatgatgtaaaaaactggactccagagttgcaaaagtatttaaactttgatccccgaacagctcagaaaatagacaacggaatattttggatttcctgggatgatctctgccagtattatgatgtgatttatttgagttggaatccaggtctttttaaagaatcaacatgtattcacagtacttgggatgctaagcaaggacctgtgaaagatgcctatagcctggccaacaacccccagtacaaactggaggtgcagtgtccacaggggggtgctgcagtttgggttttgcttagtagacacataacagacaaggatgattttgcgaataatcgagaatttatcacaatggttgtatacaagactgatgggaaaaaagtttattacccagctgacccacctccatacattgatggaattcgaattaacagccctcattatttgactaagataaagctgaccacacctggcacccatacctttacattagtggtttctcaatatgaaaaacagaacacaatccattacacggttcgggtatattcagcatgcagctttactttttcaaagattccttcaccatacaccttatcaaaacggattaatggaaagtggagtggtcagagtgctggaggatgtggaaatttccaagagactcacaaaaataaccccatctaccaattccatatagaaaagactgggccgttactgattgagctacgaggaccaaggcaatatagcgttggatttgaggttgtaacagtttctactctaggagatcctggtccccatggctttctgaggaaatctagtggtgactataggtgtgggttttgctacctggaattagaaaatataccttctgggatcttcaatatcattcctagtacctttttgcctaaacaagaaggaccttttttcttggactttaatagtattatccccatcaagatcacacaacttcagtgatggagaaatctcaagttactggcttttatacttaccaaacatcagttcttcaaataaggacgcaaatcttcaggacagtaagcagaacaatcagaatggaattaaatctctaaaaacgtgttacagtggaatctggtgcttgtcagggtgtttggtaagaactgtatatagtcagaattacctaaatcacctagaggtaccgtttacatggttttgtgtatatagagttggcttgcattttaggggccattttgtataaaaagtgcatatgattaaaattagactcagtcatcactgtgagatgcctttgctaagaggataaaggaactgagaccagatgagaaaaagaaaggatatagattccttgagtggaatagtgggctagattaatataccgaaatatttccattgtttcccttttttgcagagcatgtggaagttaaacctgcttgattctactatacatcttgggcaactagttaccaaatgaattgtgccaccataactgattttaattttgcattatttatgattttaaaatatttgttgcccaggtgttatgaaagaataaagcttttaagtatagactaccttagcatgaagatgctcatgcctaagaatgaaaattgttgaggttatctcccattcaatcatgtagcaagaacttaaagaaattcactactgcagtttttatttttaaaaaaacagtaattgagatattgaagacattacaatttagtttgtgtggtctttttttaaattgctgtatcgttcagtctcttgtggcaatagcactttgaagaaaatagagaatttaatatatggtgattgggatatgtagcattcaaaaaaangtgaattgccaagatactggtgtcatgtaaattcccactttacataaaaacccatcaggacagaatgatgctcaatattttaaaattctaaaaatagggtgggatttttcattgtctctactttataattatcaaaacttattttgtattgctactaccttaaattgaaataaaatgtttatacttaaaaaaaaaaaaaaaaaaaaaagggcggccgctagactagtctagagaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccgggtaccgagctcgaattaattcctcttccgcttcctcgctcactgactcgctgcgctcggtcgttcggctgcggcgagcggtatcagctcactcaaaggcggtaatacggttatccacagaatcaggggataacgcaggaaanaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgctttaccggatacctgtccgcctttntccctttggggaagcggnggc

SEQ ID NO: 2 - protein, 813 aa, Homo sapiens
MDATALERDAVQFARLAVQRDHEGRYSEAVFYYKEAAQALIYAEMAGSSLENIQEKITEYLERVQALHSAVQSKSADPLKSKHQLDLERAHFLVTQAFDEDEKENVEDAIELYTEAVDLCLKTSYETADKVLQNKLKQLARQALDRAEALSEPLTKPVGKISSTSVKPKPPPVRAHFPLGANPFLERPQSFISPQSCDAQGQRYTAEEIEVLRTTSKINGIEYVPFMNVDLRERFAYPMPFCDRWGKLPLSPKQKTTFSKWVRPEDLTNNPTMIYTVSSFSIKQTIVSDCSFVASLAISAAYERRFNKKLITGIIYPQNKDGEPEYNPCGKYMVKLHLNGVPRKVIIDDQLPVDHKGELLCSYSNNKSELWVSLIEKAYMKVMGGYDFPGSNSNIDLHALTGWIPERIAMHSDSQTFSKDNSFRMLYQRFHKGDVLITASTGMMTEAEGEKWGLVPTHAYAVLDIREFKGLRFIQLKNPWSHLRWKGRYSENDVKNWTPELQKYLNFDPRTAQKIDNGIFWISWDDLCQYYDVIYLSWNPGLFKESTCIHSTWDAKQGPVKDAYSLANNPQYKLEVQCPQGGAAVWVLLSRHITDKDDFANNREFITMVVYKTDGKKVYYPADPPPYIDGIRINSPHYLTKIKLTTPGTHTFTLVVSQYEKQNTIHYTVRVYSACSFTFSKIPSPYTLSKRINGKWSGQSAGGCGNFQETHKNNPIYQFHIEKTGPLLIELRGPRQYSVGFEVVTVSTLGDPGPHGFLRKSSGDYRCGFCYLELENIPSGIFNIIPSTFLPKQEGPFFLDFNSIIPIKITQLQ

(19) United States
(12) Patent Application Publication
(10) Pub. No.: US 2003/0003477 A1
(43) Pub. Date: Jan. 2, 2003

(54) 26176, a novel calpain protease and uses thereof

(75) Inventors: Rosana Kapeller-Libermann, Chestnut Hill, MA (US); Mark Williamson, Saugus, MA (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Millennium Pharmaceuticals, Inc.

(21) Appl. No.: 10/105,989

(22) Filed: Mar. 25, 2002

Related U.S. Application Data
(63) Continuation of application No. 09/392,189, filed on Sep. 9, 1999, now abandoned.

(51) Int. Cl. C12Q 1/68; C07H 21/04; C12N 9/64; C12P 21/02; C12N 5/06

(52) U.S. Cl. 435/6; 435/69.1; 435/226; 435/320.1; 435/325; 536/23.2

(57) ABSTRACT

Novel calpain protease polypeptides, proteins, and nucleic acid molecules are disclosed. In addition to isolated, full-length calpain protease proteins, the invention further provides isolated calpain protease fusion proteins, antigenic peptides, and anti-calpain protease antibodies. The invention also provides calpain protease nucleic acid molecules, recombinant expression vectors containing a nucleic acid molecule of the invention, host cells into which the expression vectors have been introduced, and nonhuman transgenic animals in which a calpain protease gene has been introduced or disrupted. Diagnostic, screening, and therapeutic methods utilizing compositions of the invention are also provided.

### CROSS-REFERENCE TO RELATED APPLICATIONS

This application is a continuation of U.S. Utility Application 09/392,189, filed Sep. 9, 1999, which is hereby incorporated in its entirety by reference herein.

### FIELD OF THE INVENTION

The invention relates to novel calpain protease nucleic acid sequences and proteins. Also provided are vectors, host cells, and recombinant methods for making and using the novel molecules.

### BACKGROUND OF THE INVENTION

Calpains refer to calcium-activated neutral proteinases, a superfamily of endopeptidases typically having cysteine-proteinase and calcium-binding characteristics. These proteinases cleave numerous substrate proteins in a limited manner, typically leading to modification of the function and/or activity rather than general degradation of the substrate.

Calpains are classified into two main groups, the typical or conventional calpains and the atypical calpains, based on their domain content and/or variation. The typical calpains are further subdivided into ubiquitous and tissue-specific calpains based on their predominate patterns of expression.

Two forms of ubiquitous calpains have been extensively characterized in vertebrates: the μ-calpains (calpain I, CAPN1) and the m-calpains (calpain II, CAPN2), which are activated in vitro by micro- and millimolar calcium concentrations, respectively. An intermediate μ/m calpain has been characterized in chicken.

The ubiquitous μ- and m-calpains are heterodimers, each having a distinct, but homologous, large 80 kDa subunit (referred to as μCL or mCL, respectively) and an identical small 30 kDa subunit (referred to as 30K or Cs). The large subunit has four domains, designated I-IV from the N-terminus to the C-terminus. The function of domain I is unclear. Domain III is the cysteine protease domain responsible for calpain protease activity. Domain III is homologous to a calmodulin-binding protein and is speculated to interact with the calcium-binding domains of the large (domain IV) and small subunits (domain VI), when calcium is bound, thereby freeing the protease domain for activity (Goll et al. (1992) BioEssays 14:549-556). Domain IV of the large subunit is a calmodulin-like calcium-binding domain containing four EF-hand calcium-binding motifs. Although structurally similar to calmodulin, domain IV is more similar to sorcin, ALG-2, and grancalcin. Sorcin is involved in the multi-drug resistance of cultured cell lines and was recently reported to associate with the cardiac ryanodine receptor. Grancalcin possibly plays a role in granule-membrane fusion and degranulation. ALG-2 is thought to be involved in apoptosis and is induced by tumor promoters. See Meyers et al. (1995) J. Biol. Chem. 270:26411-26418; Meyers et al. (1985) J. Cell Biol. 100:588-597; Vito et al. (1996) Science 271:521-525; Teahan et al. (1992) Biochem. J. 286:549-554; Boyhan et al. (1992) J. Biol. Chem. 267:2928-2933.

The large subunit of calpains is the catalytic subunit. Three non-contiguous amino acid residues, Cys, His, and Asn, residing within domain II are part of the active site. A recombinant calpain consisting essentially of domains I, II, and III showed calcium-independent activity. Thus, it has been concluded that domain II, but not IV, is necessary and sufficient for protease activity. See Vilei et al. (1997) J. Biol. Chem. 272:25802-25808; and Suzuki et al. (1998) FEBS Letters 433(1, 2):1-4.

The small subunit of typical calpains contains two domains, which are designated V and VI from the N-terminus to the C-terminus. Domain V is an N-terminal glycine-clustering hydrophobic region. Domain VI, which is similar to domain IV of the large subunit, is also a calcium-binding domain containing six EF-hands, EF2-EF5 as in the large subunit, and EF1 and EF6. EF5 of domain VI does not bind calcium and is proposed to be involved in the heterodimeric binding of domains IV and VI during interaction between the large and small subunits.

Not all calpains contain a small subunit, which is identified as a regulator of calpain activity by acting as an inhibitor or pseudosubstrate. In heterodimeric calpains, the small subunit may regulate the calcium-sensitivity of calpain by association and dissociation (Yoshizawa et al. (1995) Biochem. Biophys. Res. Commun. 208:376-383). However, the subunits remain associated during catalysis (Zhang et al. (1996) Biochem. Biophys. Res. Commun. 227:890-896).

The mechanism of activation of calpains is not entirely clear. Suggested mechanisms include combinations of N-terminal autolysis of subunits, homo- and heterodimer association/dissociation, the ratio and binding status of calpains to the calpain endogenous inhibitor calpastatin, calcium presence and concentration, and the redox state of the active site. See Johnson et al. (1997) BioEssays 19(11):1011-1018.

Because μ- and m-calpain are activated by in vitro calcium concentrations significantly above physiological levels, in vivo mechanisms that lower the calcium requirement have been proposed. Such mechanisms include interactions with membrane phospholipids and/or membrane associated proteins. See Inomata et al. (1990) Biochem. Biophys. Res. Comm. 171:625-632; and Inomata et al. (1995) Biochim. Biophys. Acta. 1235:107-114.

An activator protein specific for rat brain μ-calpain has been isolated and sequenced by Melloni et al. (1998) J. Biol. Chem. 273:12827-12831. Another activator protein specific for m-calpain is found in skeletal muscle. In addition, phospholipids, especially acidic phospholipids, have been found to greatly reduce the calcium concentration necessary for activation. Other activators and factors including DNA have been reported (Mellgren (1991) J. Biol. Chem. 266:13920-13924).

Calpastatin is an endogenous inhibitor of most calpains, the tissue-specific calpain p94 being an exception. Calpastatin, which has five domains, is cleaved by calpain in the interdomain regions, generating inhibitory peptides. The inhibitory effect of calpastatin has been attributed to interactions with calpain domains II, III, IV, and VI. The reactive site of calpastatin shows no apparent homology to that of other protease inhibitors, and it contains the consensus sequence TIPPXYR, which is essential for inhibition. See Kawasaki et al. (1989) J. Biochem. 106:274-281; Croall et al. (1994) Biochem. 33:13223-13230; Croall et al. (1991) Physiol. Rev. 71:813-847; Kawasaki et al. (1996) Mol. Membr. Biol. 13:217-224; Melloni et al. (1989) Trends Neurosci. 12:438-444; Sorimachi et al. (1997) J. Biochem. 328:721-732; and Johnson et al. (1997) BioEssays 19(11):1011-1018.

Synthetic active-site inhibitors with varying specificities for calpain and other cysteine proteases include E-64 and derivatives of E-64; leupeptin (N-acetyl-Leu-Leu -argininal); calpain inhibitors I (N-acetyl-Leu-Leu-norleucinal) and II (N-acetyl-Leu-Leu -methioninal); oxoamide inhibitor molecules AK295, AK275, and CX275; and derivatives of peptidyl α-oxo compounds. In contrast to these active-site inhibitors, PD 150606 inhibits calpains by binding the calcium-binding domains. The combination of PD 150606 and an active site inhibitor such as AK295 can inhibit calpain with high specificity. See Figueiredo-Pereira et al. (1994) J. Neuro. Chem. 62:1989-1994); Tsubuki et al. (1996) J. Biochem. (Tokyo) 119:572-576); and Sorimachi et al (1997) J. Biochem. 328:721-732.

Several typical tissue-specific calpains are known in vertebrates, including skeletal muscle p94 (nCL-1, calpain 3′, CAPN3), stomach nCL2 (CAPN4) and nCL 2′, and digestive tubule nCL4. While p94 contains EF hands, it does not require calcium for proteinase activity. p94 has a domain IV sequence similar to that of μCL and mCL, but it does not bind to a small 30 kDa subunit (Kinbara et al. (1997) Arch. Biochem. Biophys. 342:99-107). p94 contains unique insertion sequences called IS1 and IS2, which are found in domain II and between domains ImI and IV, respectively). IS2 contains a nuclear-localization-signal-like basic sequence (Arg-Pro-Xaa-Lys-Lys-Lys-Lys-x-Lys -Pro). Connectin/titin binding is also attributed to IS2. p94 may change its localization in a cell-cycle dependent manner and may be involved in muscle differentiation by interacting with the MyoD family. In fact, a defect in the protease p94 is responsible for limb-girdle muscular dystrophy type 2A (LGMD2A). See Sorimachi et aL (1995) J. Biol. Chem. 270:31158-31162; Sorimachi at al. (1993) J. Biol. Chem. 268:10593-10605; Gregoriou et al. (1994) Eur. J Biochem. 223:455-464; and Belcastro et aL (1998) Mol. Cell. Biochem. 179 (1, 2):135-145.

Atypical calpains include the flngal protein PalB, the yeast PalB homologue, the Caenorhabditis elegans protein Tra-3, human CAPN5 (htra3), CAPN6, and murine CAPN7. Although atypical calpains have a cysteine protease domain homologous to domain II of the large subunit of typical calpains, they lack a calcium-binding domain in the C-terminal portion of the protein (domain IV). See Suzuki et al. (1998) FEBS Letters 433(1, 2):1-4; Sorimachi et al. (1997) J. Biochem. 328:721-732; Franz et al. (1999) Mammalian Genome 10(3):318-321; Goll et al. (1992) BioEssays 14:549-556; and Lin et al. (1997) Nature Struct. Biol. 4:539-547.

PalB, which is involved in the alkaline adaptation of Aspergillus nidulans, is unusual in that it only has a cysteine protease domain. Tra3, which is involved in the sex-determination cascade during early development, has domains similar to domains I, II, and III of the typical calpain large subunit. Human and mouse Tra3 homologues have been identified and localized to x chromosomes, suggesting a role for calpain in sex determination in mammals. See Barnes et al. (1996) EMBO J. 15:4477-4484; and Sorimachi et al. (1997) J. Biochem. 328:721-732.

The atypical mammalian calpains include CAPN5, 6, and 7. CAPN6 and 7 contain distinct T domains in their C-terminal regions and may not associate with small subunits. These T domains have no significant homology to the cahnodulin-like calcium-binding C-terminal domain of other calpains. Furthermore, CAPN6 lacks residues believed to be critical for the active site and may lack protease activity. See Franz et al. (1999) Mammalian Genome 10(3):318-321.

Calpains have broad physiological and pathological roles related to the enzymes' diverse population of substrates. Calpain substrates include “PEST” proteins, which have high proline, glutamine, serine, and threonine contents; calpain and calpastatin; signal transduction proteins including protein kinase C, transcription factors c-Jun, c-Fos, and α-subunit of heterotrimeric G proteins; proteins involved in cell proliferation and cancer including P53 tumor suppressor, growth factor receptors (eg., epidermal growth factor receptor), c-Jun, c-Fos, and N-myc; proteins with established physiological roles in muscle including Ca⁺⁺-ATPase, Band III, troponin, tropomyosin, and myosin light chain kinase; myotonin protein kinase; proteins with established physiological roles in the brain and the central nervous system including myelin proteins, myelin basic protein (MBP), axonal neurofilament protein (NFP), myelin protein MAG; cytosketetal and cell adhesion proteins including troponins, talin, neurofilarnents, spectrin, microtubule associated protein MAP-2, tau, MAPIB, fodrin, desmin, a-actinin, vimentin, spectrin, integrin, cadherin, filamin, and N-CAM; enzymes including protein kinases A and C, and phospholipase C; and histones.

See Sorimachi et al. (1997) J. Biochem. 328:721-732; Johnson et al. (1997) BioEssays 19(11):1011-1018; Shields et al. (1999) J. NeuroscienceRes. 55(5):533-541; and Belcastro et al. (1998) Mol. Cell. Biochem. 179 (1, 2):135-145.

Calpain is implicated in a wide variety of physiological processes including alteration of membrane morphology, long-term potentiation of memory, axonal regeneration, neurite extension, cell proliferation (division), gastric HCl secretion, embryonic development, secretory granule movement, cell differentiation and regulation, cytoskelet al and membrane changes during cell migration, cytoskelet al remodeling, sex determination, and alkaline adaptation in fumgi. See Solary et al. (1998) Cell Biol. Toxicol. 14:121-132; Sorimachi et al. (1997) J. Biochem. 328:721-732; Johnson et al. (1997) BioEssays 19(11):1011-1018; Suzuki et al. (1998) FEBS Letters 433(1, 2):1-4; Franz et al. (1999) Mammalian Genome 10(3):318-321; Shields et al. (1999) J. Neuroscience Res. 55(5):533-541; Schnellmann et al. (1998) Renal Failure 20(5):679-686; Banik et al. (1998) Annals New York Acad. Sci. 844:131-137; Belcastro et al. (1998) Mol. Cell. Biochem. 179 (1, 2):135-145; and McIntosh et al. (1998) J. Neurotrauma 15(10):731-769.

Under pathological conditions, aberrant regulation and/or activity of calpain can be detrimental to cells and tissues. In this context, calpains are implicated in a wide variety of disease states including exercise-induced injury and repair; apoptosis including T cell receptor-induced apoptosis, HIV-infected cell apoptosis, ectoposide-treated cell apoptosis, nerve growth factor deprived neuronal apoptosis; ischemia, such as cerebral and myocardial ischemia; traumatic brain injury; Alzheimer's disease and other neurodegenerative diseases; demyelinating diseases including experimental allergic encephalomyelitis (EAE) and multiple sclerosis; LGMD2A muscular dystrophy; spinal cord injury (SCI); cancer; cataract formation; and renal cell death by diverse toxicants.

Given the diversity of calpains in cellular processes and disease states, compositions and methods directed to calpains are useful to influence calpain activity in a variety of tissues, thereby extending protection to cells and tissues affected with aberrant calpain function and/or regulation.

### SUMMARY OF THE INVENTION

Isolated nucleic acid molecules corresponding to calpain protease nucleic acid sequences are provided. Additionally, amino acid sequences corresponding to the polynucleotides are encompassed. In particular, the present invention provides for isolated nucleic acid molecules comprising nucleotide sequences encoding the amino acid sequence shown in SEQ ID NO:2 or the nucleotide sequences encoding the DNA sequence deposited in a bacterial host with the ATCC as Patent Deposit Number PTA-1649. Further provided are calpain protease polypeptides having an amino acid sequence encoded by a nucleic acid molecule described herein.

The present invention also provides vectors and host cells for recombinant expression of the nucleic acid molecules described herein, as well as methods of making such vectors and host cells and for using them for production of the polypeptides or peptides of the invention by recombinant techniques.

Another aspect of this invention features isolated or recombinant calpain protease proteins and polypeptides. Preferred calpain protease proteins and polypeptides possess at least one biological activity possessed by naturally occurring calpain protease proteins.

Variant nucleic acid molecules and polypeptides substantially homologous to the nucleotide and amino acid sequences set forth in the sequence listing are encompassed by the present invention. Additionally, fragments and substantially homologous fragments of the nucleotide and amino acid sequences are provided.

Antibodies and antibody fragments that selectively bind the calpain protease polypeptides and fragments are provided. Such antibodies are useful in detecting the calpain protease polypeptides as well as in regulating the T-cell immune response and cellular activity, particularly growth and proliferation.

In another aspect, the present invention provides a method for detecting the presence of calpain protease activity or expression in a biological sample by contacting the biological sample with an agent capable of detecting an indicator of calpain protease activity such that the presence of calpain protease activity is detected in the biological sample.

In yet another aspect, the invention provides a method for modulating calpain protease activity comprising contacting a cell with an agent that modulates (inhibits or stimulates) calpain protease activity or expression such that calpain protease activity or expression in the cell is modulated. In one embodiment, the agent is an antibody that specifically binds to calpain protease protein. In another embodiment, the agent modulates expression of calpain protease protein by modulating transcription of a calpain protease gene, splicing of a calpain protease mRNA, or translation of a calpain protease mRNA. In yet another embodiment, the agent is a nucleic acid molecule having a nucleotide sequence that is antisense to the coding strand of the calpain protease mRNA or the calpain protease gene.

In one embodiment, the methods of the present invention are used to treat a subject having a disorder characterized by aberrant calpain protease protein activity or nucleic acid expression by administering an agent that is a calpain protease modulator to the subject. In one embodiment, the calpain protease modulator is a calpain protease protein. In another embodiment, the calpain protease modulator is a calpain protease nucleic acid molecule. In other embodiments, the calpain protease modulator is a peptide, peptidomimetic, or other small molecule.

The present invention also provides a diagnostic assay for identifying the presence or absence of a genetic lesion or mutation characterized by at least one of the following: (1) aberrant modification or mutation of a gene encoding a calpain protease protein; (2) misregulation of a gene encoding a calpain protease protein; and (3) aberrant post-translational modification of a calpain protease protein, wherein a wild-type form of the gene encodes a protein with a calpain protease activity.

In another aspect, the invention provides a method for identifying a compound that binds to or modulates the activity of a calpain protease protein. In general, such methods entail measuring a biological activity of a calpain protease protein in the presence and absence of a test compound and identifying those compounds that alter the activity of the calpain protease protein.

The invention also features methods for identifying a compound that modulates the expression of calpain protease genes by measuring the expression of the calpain protease sequences in the presence and absence of the compound.

Other features and advantages of the invention will be apparent from the following detailed description and claims.

### BRIEF DESCRIPTION OF THE DRAWINGS

FIG. 1 shows the 26176 calpain protease nucleotide sequence (SEQ ID NO:1) and the deduced amino acid sequence (SEQ ID NO:2).

FIG. 2 shows an analysis of the 26176 calpain protease amino acid sequence: apturn and coil regions; hydrophilicity; amphipathic regions; flexible regions; antigenic index; and surface probability plot. These regions are useful with respect to, among other things, generating antigenic fragments.

FIG. 3 shows a 26176 calpain protease receptor hydrophobicity plot.

FIG. 4 shows an analysis of the 26176 calpain protease open reading frame for amino acids corresponding to specific functional sites in SEQ ID NO:2.

FIG. 5 shows an arrangement of markers on human chromosome 3 relative to the mapped position of the h26176 gene, 3p21-24.

FIG. 6 shows relative expression of h26176 in colon, liver, lung, and breast normal and carcinoma tissue samples.

### DETAILED DESCRIPTION OF THE INVENTION

The present invention provides isolated nucleic acid molecules comprising nucleotide sequences encoding the calpain protease polypeptide whose amino acid sequence is given in SEQ ID NO:2, or a variant or fragment of the polypeptide. A nucleotide sequence encoding the calpain protease polypeptides of the invention is set forth in SEQ ID NO:1. The sequences are members of the calpain family of thiol proteases, also referred to as the peptidase family C2.

Calpain proteases are endopeptidases whose cleavage sites are between, rather than within, functional domains. As a result, enzyme substrates of calpain proteases are usually activated rather than degraded, and other proteins are generally altered in their function rather than destroyed. Calpain proteases are generally calcium-dependent, and are thought to mediate intracellular calcium signaling. Controlled activation of these proteases apparently is central to a number of physiological processes, including, but not limited to, cyto/karyoskelet al remodeling, platelet activation, and cellular division, proliferation, development, and differentiation.

The disclosed invention relates to methods and compositions for the modulation, diagnosis, and treatment of calpain protease-mediated disorders. Such disorders include, but are not limited to, disorders associated with perturbed cellular growth and differentiation; exercise-induced injury and repair; apoptosis including T-cell receptor-induced apoptosis, HIV-infected cell apoptosis, ectoposide-treated cell apoptosis, nerve growth factor deprived neuronal apoptosis; ischemia; traumatic brain injury; Alzheimer's disease and other neurodegenerative diseases; demyelinating diseases including experimental allergic encephalomyelitis (EAE) and multiple sclerosis; LGMD2A muscular dystrophy; spinal cord injury (SCI); proliferative disorders or differentiative disorders such as cancer, e.g., melanoma, prostate cancer, cervical cancer, breast cancer, colon cancer, or sarcoma; and renal cell death associated with diverse toxicants.

The sequences of the invention find use in diagnosis of disorders involving an increase or decrease in protease expression relative to normal expression, such as a proliferative disorder, a differentiative disorder, or a developmental disorder. The sequences also find use in modulating protease-related responses. By “modulating” is intended the upregulating or downregulating of a response. That is, the compositions of the invention affect the targeted activity in either a positive or negative fashion.

One embodiment of the invention features protease nucleic acid molecules, preferably human protease molecules, which were identified based on a consensus motif or protein domain characteristic of the calpain family of thiol proteases. Specifically, a novel human gene, termed clone h26176, is provided. This sequence, and other nucleotide sequences encoding the h26176 protein or fragments and variants thereof, are referred to as “calpain protease sequences” indicating that the sequences share sequence similarity to other calpain protease genes.

The calpain protease gene designated clone h26176 was identified in a human T-cell cDNA library. Clone h26176 encodes an approximately 3.78 Kb mRNA transcript having the corresponding cDNA set forth in SEQ ID NO:1. This transcript has a 2439 nucleotide open reading frame (nucleotides 276-2714 of SEQ ID NO:1), which encodes an 813 amino acid protein (SEQ ID NO:2). MEMSAT analysis of the full-length h26176 polypeptide predicts a transmembrane segment from amino acids (aa) 286-302. Prosite program analysis was used to predict various sites within the h26176 protein. An N-glycosylation site was predicted at aa 366-369 with the actual residue being the first residue. A cAMP- and cGMP-dependent protein kinase phosphorylation site was predicted at aa 759-762 with the actual phosphorylated residue being the last residue. Protein kinase C phosphorylation sites were predicted at aa 165-167, 215-217, 251-253, 281-283, 422-424, 594-596, 668-670, 689-691, and 710-712 with the actual phosphorylated residue being the first residue. Casein kinase II phosphorylation sites were predicted at aa 4-7, 48-51, 123-126, 205-208, 373-376, 393-396, 445-448, 490-493, 523-526, 551-554, 594-597, 657-660, 748-751, and 761-764 with the actual phosphorylated residue being the first residue. Tyrosine kinase phosphorylation sites were predicted at aa 20-26 and aa 320-326 with the actual phosphorylated residue being the last. N-myristoylation sites were predicted at aa 201-206, 390-395, 453-458, 630-635, and 698-703 with the actual modified residue being the first. An amidation site was predicted at aa 614-617. The calpain protease protein h26176 possesses a calpain family cysteine protease domain (domain II), from aa 231-537, and a calpain large subunit domain ImI, from aa 685-810, as predicted by HMMer, Version 2.

The protein displays the closest similarity to the human gene designated PalBH, (Accession Numbers GPU:gi [5102944] dbj [BAA78730] (AB028639). The h26176 protein also displays similarity to the murine CAPN7 protein, approximately 93% identity and 95% overall similarity over a 768 amino acid overlap (amino acid residues 45-813 of the h26176 protein), indicating h26176 is the human ortholog of this murine protein.

A plasmid containing the h26176 cDNA insert was deposited with the Patent Depository of the American Type Culture Collection (ATCC), 10801 University Boulevard, Manassas, Va., on Apr. 6, 2000, and assigned Patent Deposit Number PTA-1649. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. 112.

The calpain protease sequences of the invention are members of a protease family of molecules having conserved fimctional features. The term “family” when referring to the proteins and nucleic acid molecules of the invention is intended to mean two or more proteins or nucleic acid molecules having sufficient amino acid or nucleotide sequence identity as defined herein. Such family members can be naturally occurring and can be from either the same or different species. For example, a family can contain a first protein of murine origin and an ortholog of that protein of human origin, as well as a second, distinct protein of human origin and a murine ortholog of that protein. Members of a family may also have common functional characteristics.

Preferred calpain protease polypeptides of the present invention have an amino acid sequence sufficiently identical to the amino acid sequence of SEQ ID NO:2. The term “sufficiently identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity. For example, amino acid or nucleotide sequences that contain a common structural domain having at least about 45%, 55%, or 65% identity, preferably 75% identity, more preferably 85%, 95%, or 98% identity are defined herein as sufficiently identical.

To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. The percent identity between two sequences can be determined using techniques similar to those described below, with or without allowing gaps. In calculating percent identity, typically exact matches are counted.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:403. BLAST nucleotide searches can be performed with the NBLAST program, score=100, word length=12, to obtain nucleotide sequences homologous to calpain protease nucleic acid molecules of the invention. BLAST protein searches can be performed with the XBLAST program, score=50, word length=3, to obtain amino acid sequences homologous to calpain protease protein molecules of the invention. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

Accordingly, another embodiment of the invention features isolated calpain protease proteins and polypeptides having a calpain protease protein activity. As used interchangeably herein, a “calpain protease protein activity”, “biological activity of a calpain protease protein”, or “functional activity of a calpain protease protein” refers to an activity exerted by a calpain protease protein, polypeptide, or nucleic acid molecule on a calpain-protease-responsive cell as determined in vivo, or in vitro, according to standard assay techniques. A calpain protease activity can be a direct activity, such as an association with or an enzymatic activity on a second protein, or an indirect activity, such as a cellular signaling activity mediated by interaction of the calpain protease protein with a second protein. In a preferred embodiment, a calpain protease activity includes at least one or more of the following activities: (1) modulating (stimulating and/or enhancing or inhibiting) cellular proliferation, differentiation, and/or function (e.g., in cells in which it is expressed, for example, cells within normal and carcinoma tissues, such as lung, liver, colon, and breast; brain and skelet al muscle cells, etc.); (2) modulating a calpain protease response; (3) modulating the entry of cells into mitosis; (4) modulating cellular differentiation; and (5) modulating cell death.

An “isolated” or “purified” calpain protease nucleic acid molecule or protein, or biologically active portion thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′nd 3′ ends of the nucleic acid) in the genomic DNA of the organism from which the nucleic acid is derived. For purposes of the invention, “isolated” when used to refer to nucleic acid molecules excludes isolated chromosomes. For example, in various embodiments, the isolated calpain protease nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A calpain protease protein that is substantially free of cellular material includes preparations of calpain protease protein having less than about 30%, 20%, 10%, or 5% (by dry weight) of non-calpain protease protein (also referred to herein as a “contaminating protein”). When the calpain protease protein or biologically active portion thereof is recombinantly produced, preferably, culture medium represents less than about 30%, 20%, 10%, or 5% of the volume of the protein preparation. When calpain protease protein is produced by chemical synthesis, preferably the protein preparations have less than about 30%, 20%, 10%, or 5% (by dry weight) of chemical precursors or non-calpain protease chemicals.

Various aspects of the invention are described in further detail in the following subsections.

I. Isolated Nucleic Acid Molecules

One aspect of the invention pertains to isolated nucleic acid molecules comprising nucleotide sequences encoding calpain protease proteins and polypeptides or biologically active portions thereof, as well as nucleic acid molecules sufficient for use as hybridization probes to identify calpain protease -encoding nucleic acids (e.g., calpain protease mRNA) and fragments for use as PCR primers for the amplification or mutation of calpain protease nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

Nucleotide sequences encoding the calpain protease proteins of the present invention include sequences set forth in SEQ ID NO:1, the nucleotide sequence of the cDNA insert of the plasmid deposited with the ATCC as Patent Deposit Number PTA-1649 (the “cDNA of Patent Deposit Number PTA-1649”), and complements thereof. By “complement” is intended a nucleotide sequence that is sufficiently complementary to a given nucleotide sequence such that it can hybridize to the given nucleotide sequence to thereby form a stable duplex. The corresponding amino acid sequence for the calpain protease protein encoded by these nucleotide sequences is set forth in SEQ ID NO:2.

Nucleic acid molecules that are fragments of these calpain protease nucleotide sequences are also encompassed by the present invention. By “fragment” is intended a portion of the nucleotide sequence encoding a calpain protease protein. A fragment of a calpain protease nucleotide sequence may encode a biologically active portion of a calpain protease protein, or it may be a fragment that can be used as a hybridization probe or PCR primer using methods disclosed below. A biologically active portion of a calpain protease protein can be prepared by isolating a portion of one of the calpain protease nucleotide sequences of the invention, expressing the encoded portion of the calpain protease protein (e.g., by recombinant expression in vitro), and assessing the activity of the encoded portion of the calpain protease protein. Nucleic acid molecules that are fragments of a calpain protease nucleotide sequence comprise at least 15, 20, 50, 75, 100, 200, 300, 350, 400, 450, 500, 550, 600, 650, 700, 750, 800, 850, 900, 950, 1000, 1100, 1200, 1300, 1400, 1500, 1750, 2000, 2250, 2500, 2750, 3000, 3250, 3500, 3750 nucleotides, or up to the number of nucleotides present in a full-length calpain protease nucleotide sequence disclosed herein (for example, 3777 nucleotides for SEQ ID NO:1) depending upon the intended use.

It is understood that isolated fragments include any contiguous sequence not disclosed prior to the invention as well as sequences that are substantially the same and which are not disclosed. Accordingly, if an isolated fragment is disclosed prior to the present invention, that fragment is not intended to be encompassed by the invention. When a sequence is not disclosed prior to the present invention, an isolated nucleic acid fragment is at least about 12, 15, 20, 25, or 30 contiguous nucleotides. Other regions of the nucleotide sequence may comprise fragments of various sizes, depending upon potential homology with previously disclosed sequences.

A fragment of a calpain protease nucleotide sequence that encodes a biologically active portion of a calpain protease protein of the invention will encode at least 15, 25, 30, 50, 75, 100, 125, 150, 175, 200, 250, 300, 350, 400, 450, 500, 550, 600, 650, 700, 750, 800 contiguous amino acids, or up to the total number of amino acids present in a full-length calpain protease protein of the invention (for example, 813 amino acids for SEQ ID NO:2). Fragments of a calpain protease nucleotide sequence that are useful as hybridization probes for PCR primers generally need not encode a biologically active portion of a calpain protease protein.

Nucleic acid molecules that are variants of the calpain protease nucleotide sequences disclosed herein are also encompassed by the present invention. “Variants” of the calpain protease nucleotide sequences include those sequences that encode the calpain protease proteins disclosed herein but that differ conservatively because of the degeneracy of the genetic code. These naturally occurring allelic variants can be identified with the use of well-known molecular biology techniques, such as polymerase chain reaction (PCR) and hybridization techniques as outlined below. Variant nucleotide sequences also include synthetically derived nucleotide sequences that have been generated, for example, by using site-directed mutagenesis but which still encode the calpain protease proteins disclosed in the present invention as discussed below. Generally, nucleotide sequence variants of the invention will have at least 45%, 55%, 65%, 75%, 85%, 95%, or 98% identity to a particular nucleotide sequence disclosed herein. A variant calpain protease nucleotide sequence will encode a calpain protease protein that has an amino acid sequence having at least 45%, 55%, 65%, 75%, 85%, 95%, or 98% identity to the amino acid sequence of a calpain protease protein disclosed herein.

In addition to the calpain protease nucleotide sequence shown in SEQ ID NO:1, and the nucleotide sequence of the cDNA of Patent Deposit Number PTA-1649, it will be appreciated by those skilled in the art that DNA sequence polymorphisms that lead to changes in the amino acid sequences of calpain protease proteins may exist within a population (e.g., the human population). Such genetic polymorphism in a calpain protease gene may exist among individuals within a population due to natural allelic variation. An allele is one of a group of genes that occur alternatively at a given genetic locus. As used herein, the terms “gene” and “recombinant gene” refer to nucleic acid molecules comprising an open reading frame encoding a calpain protease protein, preferably a mammalian calpain protease protein. As used herein, the phrase “allelic variant” refers to a nucleotide sequence that occurs at a calpain protease locus or to a polypeptide encoded by the nucleotide sequence. Such natural allelic variations can typically result in 1-5% variance in the nucleotide sequence of the calpain protease gene. Any and all such nucleotide variations and resulting amino acid polymorphisms or variations in a calpain protease sequence that are the result of natural allelic variation and that do not alter the functional activity of calpain protease proteins are intended to be within the scope of the invention.

Moreover, nucleic acid molecules encoding calpain protease proteins from other species (calpain protease homologues), which have a nucleotide sequence differing from that of the calpain protease sequences disclosed herein, are intended to be within the scope of the invention. For example, nucleic acid molecules corresponding to natural allelic variants and homologues of the human calpain protease cDNA of the invention can be isolated based on their identity to the human calpain protease nucleic acid disclosed herein using the human cDNA, or a portion thereof, as a hybridization probe according to standard hybridization techniques under stringent hybridization conditions as disclosed below.

In addition to naturally-occurring allelic variants of the calpain protease sequences that may exist in the population, the skilled artisan will further appreciate that changes can be introduced by mutation into the nucleotide sequences of the invention thereby leading to changes in the amino acid sequence of the encoded calpain protease proteins, without altering the biological activity of the calpain protease proteins. Thus, an isolated nucleic acid molecule encoding a calpain protease protein having a sequence that differs from that of SEQ ID NO:2 can be created by introducing one or more nucleotide substitutions, additions, or deletions into the corresponding nucleotide sequence disclosed herein, such that one or more amino acid substitutions, additions or deletions are introduced into the encoded protein. Mutations can be introduced by standard techniques, such as site-directed mutagenesis and PCR-mediated mutagenesis. Such variant nucleotide sequences are also encompassed by the present invention.

For example, preferably, conservative amino acid substitutions may be made at one or more predicted, preferably nonessential amino acid residues. A “nonessential” amino acid residue is a residue that can be altered from the wild-type sequence of a calpain protease protein (e.g., the sequence of SEQ ID NO:2) without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Such substitutions would not be made for conserved amino acid residues, or for amino acid residues residing within a conserved motif, such as the calpain family cysteine protease domain (aa residues 231-537 of SEQ ID NO:2) or calpain large subunit domain III (aa residues 685-810 of SEQ ID NO:2), where such residues are essential for protein activity.

Alternatively, variant calpain protease nucleotide sequences can be made by introducing mutations randomly along all or part of a calpain protease coding sequence, such as by saturation mutagenesis, and the resultant mutants can be screened for calpain protease biological activity to identify mutants that retain activity. Following mutagenesis, the encoded protein can be expressed recombinantly, and the activity of the protein can be determined using standard assay techniques.

Thus the nucleotide sequences of the invention include the sequences disclosed herein as well as fragments and variants thereof. The calpain protease nucleotide sequences of the invention, and fragments and variants thereof, can be used as probes and/or primers to identify and/or clone calpain protease homologues in other cell types, e.g., from other tissues, as well as calpain protease homologues from other mammals. Such probes can be used to detect transcripts or genomic sequences encoding the same or identical proteins. These probes can be used as part of a diagnostic test kit for identifying cells or tissues that misexpress a calpain protease protein, such as by measuring levels of a calpain protease-encoding nucleic acid in a sample of cells from a subject, e.g., detecting calpain protease mRNA levels or determining whether a genomic calpain protease gene has been mutated or deleted.

In this manner, methods such as PCR, hybridization, and the like can be used to identify such sequences having substantial identity to the sequences of the invention. See, for example, Sambrook et aL (1989) Molecular Cloning: Laboratory Manual (2d ed., Cold Spring Harbor Laboratory Press, Plainview, N.Y.) and Innis, et al. (1990) PCR Protocols: A Guide to Methods and Applications (Academic Press, NY). calpain protease nucleotide sequences isolated based on their sequence identity to the calpain protease nucleotide sequences set forth herein or to fragments and variants thereof are encompassed by the present invention.

In a hybridization method, all or part of a known calpain protease nucleotide sequence can be used to screen cDNA or genomic libraries. Methods for construction of such cDNA and genomic libraries are generally known in the art and are disclosed in Sambrook et al. (1989) Molecular Cloning: A Laboratory Manual (2d ed., Cold Spring Harbor Laboratory Press, Plainview, N.Y.). The so-called hybridization probes may be and may be labeled with a detectable group such as ³²P, or any other detectable marker, such as other radioisotopes, a fluorescent compound, an enzyme, or an enzyme co-factor. Probes for hybridization can be made by labeling synthetic oligonucleotides based on the known calpain protease nucleotide sequence disclosed herein. Degenerate primers designed on the basis of conserved nucleotides or amino acid residues in a known calpain protease nucleotide sequence or encoded amino acid sequence can additionally be used. The probe typically comprises a region of nucleotide sequence that hybridizes under stringent conditions to at least about 12, preferably about 25, more preferably about 50, 75, 100, 125, 150, 175, 200, 250, 300, 350, or 400 consecutive nucleotides of a calpain protease nucleotide sequence of the invention or a fragment or variant thereof. Preparation of probes for hybridization is generally known in the art and is disclosed in Sambrook et al. (1989) Molecular Cloning: A Laboratory Manual (2d ed., Cold Spring Harbor Laboratory Press, Plainview, N.Y.), herein incorporated by reference.

For example, in one embodiment, a previously unidentified calpain protease nucleic acid molecule hybridizes under stringent conditions to a probe that is a nucleic acid molecule comprising one of the calpain protease nucleotide sequences of the invention or a fragment thereof. In another embodiment, the previously unknown calpain protease nucleic acid molecule is at least 300, 325, 350, 375, 400, 425, 450, 500, 550, 600, 650, 700, 800, 900, 1000, 2,000, 3,000, 4,000 or 5,000 nucleotides in length and hybridizes under stringent conditions to a probe that is a nucleic acid molecule comprising one of the calpain protease nucleotide sequences disclosed herein or a fragment thereof.

Accordingly, in another embodiment, an isolated previously unknown calpain protease nucleic acid molecule of the invention is at least 300, 325, 350, 375, 400, 425, 450, 500, 550, 600, 650, 700, 800, 900, 1000, 1,100, 1,200, 1,300, or 1,400 nucleotides in length and hybridizes under stringent conditions to a probe that is a nucleic acid molecule comprising one of the nucleotide sequences of the invention, preferably the coding sequence set forth in SEQ ID NO:1, the cDNA of Patent Deposit Number PTA-1649, or a complement, fragment, or variant thereof.

As used herein, the term “hybridizes under stringent conditions” is intended to describe conditions for hybridization and washing under which nucleotide sequences having at least 60%, 65%, 70%, preferably 75% identity to each other typically remain hybridized to each other. Such stringent conditions are known to those skilled in the art and can be found in Current Protocols in Molecular Biology (John Wiley & Sons, New York, 1989), 6.3.1-6.3.6. A preferred, non-limiting example of stringent hybridization conditions is hybridization in 6× sodium chloride/sodium citrate (SSC) at about 45° C., followed by one or more washes in 0.2×SSC, 0.1% SDS at 50-65° C. In another preferred embodiment, stringent conditions comprise hybridization in 6×SSC at 42° C., followed by washing with 1×SSC at 55° C. Preferably, an isolated nucleic acid molecule that hybridizes under stringent conditions to a calpain protease sequence of the invention corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

Thus, in addition to the calpain protease nucleotide sequences disclosed herein and fragments and variants thereof, the isolated nucleic acid molecules of the invention also encompass homologous DNA sequences identified and isolated from other cells and/or organisms by hybridization with entire or partial sequences obtained from the calpain protease nucleotide sequences disclosed herein or variants and fragments thereof.

The present invention also encompasses antisense nucleic acid molecules, i.e., molecules that are complementary to a sense nucleic acid encoding a protein, e.g., complementary to the coding strand of a double-stranded cDNA molecule, or complementary to an mRNA sequence. Accordingly, an antisense nucleic acid can hydrogen bond to a sense nucleic acid. The antisense nucleic acid can be complementary to an entire calpain protease coding strand, or to only a portion thereof, e.g., all or part of the protein coding region (or open reading frame). An antisense nucleic acid molecule can be antisense to a noncoding region of the coding strand of a nucleotide sequence encoding a calpain protease protein. The noncoding regions are the 5′ and 3′ sequences that flank the coding region and are not translated into amino acids.

Given the coding-strand sequence encoding a calpain protease protein disclosed herein (e.g., SEQ ID NO:1), antisense nucleic acids of the invention can be designed according to the rules of Watson and Crick base pairing. The antisense nucleic acid molecule can be complementary to the entire coding region of calpain protease mRNA, but more preferably is an oligonucleotide that is antisense to only a portion of the coding or noncoding region of calpain protease mRNA. For example, the antisense oligonucleotide can be complementary to the region surrounding the translation start site of calpain protease mRNA. An antisense oligonucleotide can be, for example, about 5, 10, 15, 20, 25, 30, 35, 40, 45, or 50 nucleotides in length. An antisense nucleic acid of the invention can be constructed using chemical synthesis and enzymatic ligation procedures known in the art.

For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, including, but not limited to, for example e.g., phosphorothioate derivatives and acridine substituted nucleotides. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a calpain protease protein to thereby inhibit expression of the protein, e.g., by inhibiting transcription and/or translation. An example of a route of administration of antisense nucleic acid molecules of the invention includes direct Iinjection at a tissue site. Alternatively, antisense nucleic acid molecules can be modified to target selected cells and then administered systemically. For example, antisense molecules can be linked to peptides or antibodies to form a complex that specifically binds to receptors or antigens expressed on a selected cell surface. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an x-anomeric nucleic acid molecule. An α-anomeric nucleic acid molecule forms specific double-stranded hybrids with complementary RNA in which, contrary to the usual P-units, the strands run parallel to each other (Gaultier et al. (1987) Nucleic Acids Res. 15:6625-6641). The antisense nucleic acid molecule can also comprise a 2′-o-methylribonucleotide (lnoue et al. (1987) Nucleic Acids Res. 15:6131-6148) or a chimeric RNA-DNA analogue (Inoue et al. (1987) FEBS Lett. 215:327-330).

The invention also encompasses ribozymes, which are catalytic RNA molecules with ribonuclease activity that are capable of cleaving a single-stranded nucleic acid, such as an mRNA, to which they have a complementary region. Ribozymes (e.g., hammerhead ribozymes (described in Haselhoff and Gerlach (1988) Nature 334:585-591)) can be used to catalytically cleave calpain protease mRNA transcripts to thereby inhibit translation of calpain protease mRNA. A ribozyme having specificity for a calpain protease -encoding nucleic acid can be designed based upon the nucleotide sequence of a calpain protease cDNA disclosed herein (e.g., SEQ ID NO:1). See, e.g., Cech et al., U.S. Pat. No. 4,987,071; and Cech et al., U.S. Pat. No. 5,116,742. Alternatively, calpain protease mRNA can be used to select a catalytic RNA having a specific ribonuclease activity from a pool of RNA molecules. See, e.g., Bartel and Szostak (1993) Science 261:1411-1418.

The invention also encompasses nucleic acid molecules that form triple helical structures. For example, calpain protease gene expression can be inhibited by targeting nucleotide sequences complementary to the regulatory region of the calpain protease protein (e.g., the calpain protease promoter and/or enhancers) to form triple helical structures that prevent transcription of the calpain protease gene in target cells. See generally Helene (1991) Anticancer Drug Des. 6(6):569; Helene (1992) Ann. N.Y Acad. Sci. 660:27; and Maher (1992) Bioassays 14(12):807.

In preferred embodiments, the nucleic acid molecules of the invention can be modified at the base moiety, sugar moiety, or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4:5). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid-phase peptide synthesis protocols as described in Hyrup et al. (1996), supra; Perry-O'Keefe et al. (1996) Proc. Natl. Acad. Sci. USA 93:14670.

PNAs of a calpain protease molecule can be used in therapeutic and diagnostic applications. For example, PNAs can be used as antisense or antigene agents for sequence-specific modulation of gene expression by, e.g., inducing transcription or translation arrest or inhibiting replication. PNAs of the invention can also be used, e.g., in the analysis of single base pair mutations in a gene by, e.g., PNA-directed PCR clamping; as artificial restriction enzymes when used in combination with other enzymes, e.g., S1 nucleases (Hyrup (1996), supra; or as probes or primers for DNA sequence and hybridization (Hyrup (1996), supra; Perry-O'Keefe et al. (1996), supra).

In another embodiment, PNAs of a calpain protease molecule can be modified, e.g., to enhance their stability, specificity, or cellular uptake, by attaching lipophilic or other helper groups to PNA, by the formation of PNA-DNA chimeras, or by the use of liposomes or other techniques of drug delivery known in the art. The synthesis of PNA-DNA chimeras can be performed as described in Hyrup (1996), supra; Finn et al. (1996) Nucleic Acids Res. 24(17):3357-63; Mag et al. (1989) Nucleic Acids Res. 17:5973; and Peterson et al. (1975) Bioorganic Med. Chem. Lett. 5:1119.

II. Isolated calpain protease Proteins and Anti-calpain protease Antibodies

Calpain protease proteins are also encompassed within the present invention. By “calpain protease protein” is intended a protein having the amino acid sequence set forth in SEQ ID NO:2, as well as fragments, biologically active portions, and variants thereof.

“Fragments” or “biologically active portions” include polypeptide fragments suitable for use as immunogens to raise anti-calpain protease antibodies. Fragments include peptides comprising amino acid sequences sufficiently identical to or derived from the amino acid sequence of a calpain protease protein, or partial-length protein, of the invention and exhibiting at least one activity of a calpain protease protein, but which include fewer amino acids than the full-length (SEQ ID NO:2) calpain protease protein disclosed herein. Typically, biologically active portions comprise a domain or motif with at least one activity of the calpain protease protein. A biologically active portion of a calpain protease protein can be a polypeptide which is, for example, 10, 25, 50, 100 or more amino acids in length. Such biologically active portions can be prepared by recombinant techniques and evaluated for one or more of the functional activities of a native calpain protease protein. As used here, a fragment not previously disclosed comprises at least 5 contiguous amino acids of SEQ ID NO:2. The invention encompasses other fragments, however, such as any fragment in the protein greater than 6, 7, 8, or 9 amino acids that has not been previously disclosed.

By “variants” is intended proteins or polypeptides having an amino acid sequence that is at least about 45%, 55%, 65%, preferably about 75%, 85%, 95%, or 98% identical to the amino acid sequence of SEQ ID NO:2. Variants also include polypeptides encoded by the cDNA insert of the plasmid deposited with ATCC as Patent Deposit Number PTA-1649, or polypeptides encoded by a nucleic acid molecule that hybridizes to the nucleic acid molecule of SEQ ID NO:1, or a complement thereof, under stringent conditions. Such variants generally retain the functional activity of the calpain protease proteins of the invention. Variants include polypeptides that differ in amino acid sequence due to natural allelic variation or mutagenesis.

The invention also provides calpain protease chimeric or fusion proteins. As used herein, a calpain protease “chimeric protein” or “fusion protein” comprises a calpain protease polypeptide operably linked to a non-calpain protease polypeptide. A “calpain protease polypeptide” refers to a polypeptide having an amino acid sequence corresponding to a calpain protease protein, whereas a “non-calpain protease polypeptide” refers to a polypeptide having an amino acid sequence corresponding to a protein that is not substantially identical to the calpain protease protein, e.g., a protein that is different from the calpain protease protein and which is derived from the same or a different organism. Within a calpain protease fusion protein, the calpain protease polypeptide can correspond to all or a portion of a calpain protease protein, preferably at least one biologically active portion of a calpain protease protein. Within the fusion protein, the term “operably linked” is intended to indicate that the calpain protease polypeptide and the non-calpain protease polypeptide are fused in-frame to each other. The non-calpain protease polypeptide can be fused to the N-terminus or C-terminus of the calpain protease polypeptide.

One useful fusion protein is a GST-calpain protease fusion protein in which the calpain protease sequences are fused to the N- or C-terminus of the GST sequences. Such fusion proteins can facilitate the purification of recombinant calpain protease proteins.

In yet another embodiment, the fusion protein is a calpain protease-immunoglobulin fusion protein in which all or part of a calpain protease protein is fused to sequences derived from a member of the immunoglobulin protein family. The calpain protease-immunoglobulin fusion proteins of the invention can be incorporated into pharmaceutical compositions and administered to a subject to inhibit an interaction between a calpain protease ligand and a calpain protease protein on the surface of a cell, thereby suppressing calpain protease-mediated signal transduction in vivo. The calpain protease-immunoglobulin fusion proteins can be used to affect the bioavailability of a calpain protease cognate ligand. Inhibition of the calpain protease ligand/calpain protease interaction may be useful therapeutically, both for treating proliferative and differentiative disorders and for modulating (e.g., promoting or inhibiting) cell survival. Moreover, the calpain protease-immunoglobulin fusion proteins of the invention can be used as immunogens to produce anti-calpain protease antibodies in a subject, to purify calpain protease ligands, and in screening assays to identify molecules that inhibit the interaction of a calpain protease protein with a calpain protease ligand.

Preferably, a calpain protease chimeric or fusion protein of the invention is produced by standard recombinant DNA techniques. For example, DNA fragments coding for the different polypeptide sequences may be ligated together in-frame, or the fusion gene can be synthesized, such as with automated DNA synthesizers. Alternatively, PCR amplification of gene fragments can be carried out using anchor primers that give rise to complementary overhangs between two consecutive gene fragments, which can subsequently be annealed and reamplified to generate a chimeric gene sequence (see, e.g., Ausubel et al., eds. (1995) Current Protocols in Molecular Biology) (Greene Publishing and Wiley-Interscience, NY). Moreover, a calpain protease -encoding nucleic acid can be cloned into a commercially available expression vector such that it is linked in-frame to an existing fusion moiety. Variants of the calpain protease proteins can function as either calpain protease agonists (mimetics) or as calpain protease antagonists. Variants of the calpain protease protein can be generated by mutagenesis, e.g., discrete point mutation or truncation of the calpain protease protein. An agonist of the calpain protease protein can retain substantially the same, or a subset, of the biological activities of the naturally occurring form of the calpain protease protein. An antagonist of the calpain protease protein can inhibit one or more of the activities of the naturally occurring form of the calpain protease protein by, for example, competitively binding to a downstream or upstream member of a cellular signaling cascade that includes the calpain protease protein. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the calpain protease proteins.

Variants of a calpain protease protein that function as either calpain protease agonists or as calpain protease antagonists can be identified by screening combinatorial libraries of mutants, e.g., truncation mutants, of a calpain protease protein for calpain protease protein agonist or antagonist activity. In one embodiment, a variegated library of calpain protease variants is generated by combinatorial mutagenesis at the nucleic acid level and is encoded by a variegated gene library. A variegated library of calpain protease variants can be produced by, for example, enzymatically ligating a mixture of synthetic oligonucleotides into gene sequences such that a degenerate set of potential calpain protease sequences is expressible as individual polypeptides, or alternatively, as a set of larger fusion proteins (e.g., for phage display) containing the set of calpain protease sequences therein. There are a variety of methods that can be used to produce libraries of potential calpain protease variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential calpain protease sequences. Methods for synthesizing degenerate oligonucleotides are known in the art (see, e.g., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of a calpain protease protein coding sequence can be used to generate a variegated population of calpain protease fragments for screening and subsequent selection of variants of a calpain protease protein. In one embodiment, a library of coding sequence fragments can be generated by treating a double-stranded PCR fragment of a calpain protease coding sequence with a nuclease under conditions wherein nicking occurs only about once per molecule, denaturing the double-stranded DNA, renaturing the DNA to form double-stranded DNA which can include sense/antisense pairs from different nicked products, removing single-stranded portions from reformed duplexes by treatment with S1 nuclease, and ligating the resulting fragment library into an expression vector. By this method, one can derive an expression library that encodes N-terminal and internal fragments of various sizes of the calpain protease protein.

Several techniques are known in the art for screening gene products of combinatorial libraries made by point mutations or truncation and for screening cDNA libraries for gene products having a selected property. Such techniques are adaptable for rapid screening of the gene libraries generated by the combinatorial mutagenesis of calpain protease proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique that enhances the frequency of functional mutants in the libraries, can be used in combination with the screening assays to identify calpain protease variants (Arkin and Yourvan (1992) Proc. Natl. Acad. Sci. USA 89:7811-7815; Delgrave et al. (1993) Protein Engineering 6(3):327-331).

An isolated calpain protease polypeptide of the invention can be used as an immunogen to generate antibodies that bind calpain protease proteins using standard techniques for polyclonal and monoclonal antibody preparation. The full-length calpain protease protein can be used or, alternatively, the invention provides antigenic peptide fragments of calpain protease proteins for use as immunogens. The antigenic peptide of a calpain protease protein comprises at least 8, preferably 10, 15, 20, or 30 amino acid residues of the amino acid sequence shown in SEQ ID NO:2 and encompasses an epitope of a calpain protease protein such that an antibody raised against the peptide forms a specific immune complex with the calpain protease protein. Preferred epitopes encompassed by the antigenic peptide are regions of a calpain protease protein that are located on the surface of the protein, e.g., hydrophilic regions.

Accordingly, another aspect of the invention pertains to anti-calpain protease polyclonal and monoclonal antibodies that bind a calpain protease protein. Polyclonal anti-calpain protease antibodies can be prepared by immunizing a suitable subject (e.g., rabbit, goat, mouse, or other mammal) with a calpain protease immunogen. The anti-calpain protease antibody titer in the immunized subject can be monitored over time by standard techniques, such as with an enzyme linked immunosorbent assay (ELISA) using immobilized calpain protease protein. At an appropriate time after immunization, e.g., when the anti-calpain protease antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985) in Monoclonal Antibodies and Cancer Therapy, ed. Reisfeld and Sell (Alan R. Liss, Inc., New York, N.Y.), pp. 77-96) or trioma techniques. The technology for producing hybridomas is well known (see generally Coligan et al., eds. (1994) Current Protocols in Immunology (John Wiley & Sons, Inc., New York, N.Y.); Galfre et al. (1977) Nature 266:55052; Kenneth (1980) in Monoclonal Antibodies: A New Dimension In Biological Analyses (Plenum Publishing Corp., NY; and Lerner (1981) Yale J. Biol. Med., 54:387-402).

Alternative to preparing monoclonal antibody-secreting hybridomas, a monoclonal anti-calpain protease antibody can be identified and isolated by screening a recombinant combinatorial immunoglobulin library (e.g., an antibody phage display library) with a calpain protease protein to thereby isolate immunoglobulin library members that bind the calpain protease protein. Kits for generating and screening phage display libraries are commercially available (e.g., the Pharmacia Recombinant Phage Antibody System, Catalog No. 27-9400-01; and the Stratagene SurfZAPθ Phage Display Kit, Catalog No. 240612). Additionally, examples of methods and reagents particularly amenable for use in generating and screening antibody display library can be found in, for example, U.S. Pat. No. 5,223,409; PCT Publication Nos. WO 92/18619; WO 91/17271; WO 92/20791; WO 92/15679; 93/01288; WO 92/01047; 92/09690; and 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant anti-calpain protease antibodies, such as chimeric and humanized monoclonal antibodies, comprising both human and nonhuman portions, which can be made using standard recombinant DNA techniques, are within the scope of the invention. Such chimeric and humanized monoclonal antibodies can be produced by recombinant DNA techniques known in the art, for example using methods described in PCT Publication Nos. WO 86101533 and WO 87/02671; European Patent Application Nos. 184,187, 171,496, 125,023, and 173,494; U.S. Pat. Nos. 4,816,567 and 5,225,539; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; Shaw et al. (1988) J. Natl. Cancer Inst. 80:1553-1559); Morrison (1985) Science 229:1202-1207; Oi et al. (1 986) Bio/Techniques 4:214; Jones et al. (1 986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. See, for example, Lonberg and Huszar (1995) Int. Rev. Immunol. 13:65-93); and U.S. Pat. Nos. 5,625,126; 5,633,425; 5,569,825; 5,661,016; and 5,545,806. In addition, companies such as Abgenix, Inc. (Freemont, Calif.), can be engaged to provide human antibodies directed against a selected antigen using technology similar to that described above.

Completely human antibodies that recognize a selected epitope can be generated using a technique referred to as “guided selection.” In this approach a selected non-human monoclonal antibody, e.g., a murine antibody, is used to guide the selection of a completely human antibody recognizing the same epitope. This technology is described by Jespers et al. (1994) Bio/Technology 12:899-903).

An anti-calpain protease antibody (e.g., monoclonal antibody) can be used to isolate calpain protease proteins by standard techniques, such as affinity chromatography or immunoprecipitation. An anti-calpain protease antibody can facilitate the purification of natural calpain protease protein from cells and of recombinantly produced calpain protease protein expressed in host cells. Moreover, an anti-calpain protease antibody can be used to detect calpain protease protein (e.g., in a cellular lysate or cell supernatant) in order to evaluate the abundance and pattern of expression of the calpain protease protein. Anti-calpain protease antibodies can be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidinlbiotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S, or ³H.

III. Recombinant Expression Vectors and Host Cells

Another aspect of the invention pertains to vectors, preferably expression vectors, containing a nucleic acid encoding a calpain protease protein (or a portion thereof). “Vector” refers to a nucleic acid molecule capable of transporting another nucleic acid to which it has been linked, such as a “plasmid”, a circular double-stranded DNA loop into which additional DNA segments can be ligated, or a viral vector, where additional DNA segments can be ligated into the viral genome. The vectors are useful for autonomous replication in a host cell or may be integrated into the genome of a host cell upon introduction into the host cell, and thereby are replicated along with the host genome (e.g., nonepisomal mammalian vectors). Expression vectors are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses, and adeno-associated viruses), that serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, operably linked to the nucleic acid sequence to be expressed. “Operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to include promoters, enhancers, and other expression control elements (e.g., polyadenylation signals). See, for example, Goeddel (1990) in Gene Expression Technology: Methods in Enzymology 185 (Academic Press, San Diego, Calif.). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., calpain protease proteins, mutant forms of calpain protease proteins, fusion proteins, etc.).

The recombinant expression vectors of the invention can be designed for expression of calpain protease protein in prokaryotic or eukaryotic host cells. Expression of proteins in prokaryotes is most often carried out in E. coli with vectors containing constitutive or inducible promoters directing the expression of either fusion or nonfusion proteins. Fusion vectors add a number of amino acids to a protein encoded therein, usually to the amino terminus of the recombinant protein. Typical fusion expression vectors include pGEX (Pharmacia Biotech Inc; Smith and Johnson (1988) Gene 67:31-40), pMAL (New England Biolabs, Beverly, Mass.), and pRIT5 (Pharmacia, Piscataway, N.J.) which fuse glutathione S-transferase (GST), maltose E binding protein, or protein A, respectively, to the target recombinant protein. Examples of suitable inducible nonfusion E. coli expression vectors include pTrc (Amann et al. (1988) Gene 69:301-315) and pET 11d (Studier et al. (1990) in Gene Expression Technology: Methods in Enzymology 185 (Academic Press, San Diego, Calif.), pp. 60-89). Strategies to maximize recombinant protein expression in E. coli can be found in Gottesman (1990) in Gene Expression Technology: Methods in Enzymology 185 (Academic Press, Calif.), pp. 119-128 and Wada et al. (1992) Nucleic Acids Res. 20:2111-2118. Target gene expression from the pTrc vector relies on host RNA polymerase transcription from a hybrid trp-lac fusion promoter.

Suitable eukaryotic host cells include insect cells (examples of Baculovirus vectors available for expression of proteins in cultured insect cells (e.g., Sf 9 cells) include the pAc series (Smith et al. (1983) Mol Cell Biol. 3:2156-2165) and the pVL series (Lucklow and Summers (1989) Virology 170:31-39)); yeast cells (examples of vectors for expression in yeast S. cereivisiae include pYepSec1 (Baldari et al. (1987) EMBO J. 6:229-234), pMFa (Kuijan and Herskowitz (1982) Cell 30:933-943), pJRY88 (Schultz et al. (1987) Gene 54:113-123), pYES2 (invitrogen Corporation, San Diego, Calif.), and pPicZ (Invitrogen Corporation, San Diego, Calif.)); or mammalian cells (mammalian expression vectors include pCDM8 (Seed (1987) Nature 329:840) and pMT2PC (Kaufmnan et al. (1987) EMBO J. 6:187:195)). Suitable mammalian cells include Chinese hamster ovary cells (CHO) or COS cells. In mammalian cells, the expression vector's control functions are often provided by viral regulatory elements. For example, commonly used promoters are derived from polyoma, Adenovirus 2, cytomegalovirus, and Simian Virus 40. For other suitable expression systems for both prokaryotic and eukaryotic cells, see chapters 16 and 17 of Sambrook et al. (1989) Molecular cloning: A Laboratory Manual (2d ed., Cold Spring Harbor Laboratory Press, Plainview, N.Y.). See, Goeddel (1990) in Gene Expression Technology: Methods in Enzymology 185 (Academic Press, San Diego, Calif.). Alternatively, the recombinant expression vector can be transcribed and translated in vitro, for example using T7 promoter regulatory sequences and T7 polymerase.

The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell but are still included within the scope of the term as used herein.

In one embodiment, the expression vector is a recombinant mammalian expression vector that comprises tissue-specific regulatory elements that direct expression of the nucleic acid preferentially in a particular cell type. Suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J 8:729-733) and immunoglobulins (Baneiji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Patent Publication No. 264,166). Developmentally-regulated promoters are also encompassed, for example the murine hox promoters (Kessel and Gruss (1990) Science 249:374-379), the 0:-fetoprotein promoter (Campes and Tilghman (1989) Genes Dev. 3:537-546), and the like.

The invention further provides a recombinant expression vector comprising a DNA molecule of the invention cloned into the expression vector in an antisense orientation. That is, the DNA molecule is operably linked to a regulatory sequence in a manner that allows for expression (by transcription of the DNA molecule) of an RNA molecule that is antisense to calpain protease mRNA. Regulatory sequences operably linked to a nucleic acid cloned in the antisense orientation can be chosen to direct the continuous expression of the antisense RNA molecule in a variety of cell types, for instance viral promoters and/or enhancers, or regulatory sequences can be chosen to direct constitutive, tissue-specific, or cell-type-specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. (1986) Reviews—Trends in Genetics, Vol. 1(1).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (1989) Molecular Cloning: A Laboraty Manual (2d ed., Cold Spring Harbor Laboratory Press, Plainview, N.Y.) and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin, and methotrexate. Nucleic acid encoding a selectable marker can be introduced into a host cell on the same vector as that encoding a calpain protease protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) calpain protease protein. Accordingly, the invention further provides methods for producing calpain protease protein using the host cells of the invention. In one embodiment, the method comprises culturing the host cell of the invention, into which a recombinant expression vector encoding a calpain protease protein has been introduced, in a suitable medium such that calpain protease protein is produced. In another embodiment, the method further comprises isolating calpain protease protein from the medium or the host cell.

The host cells of the invention can also be used to produce nonhuman transgenic animals. For example, in one embodiment, a host cell of the invention is a fertilized oocyte or an embryonic stem cell into which calpain protease-coding sequences have been introduced. Such host cells can then be used to create nonhuman transgenic animals in which exogenous calpain protease sequences have been introduced into their genome or homologous recombinant animals in which endogenous calpain protease sequences have been altered. Such animals are useful for studying the function and/or activity of calpain protease genes and proteins and for identifying and/or evaluating modulators of calpain protease activity. As used herein, a “transgenic animal” is a nonhuman animal, preferably a mammal, more preferably a rodent such as a rat or mouse, in which one or more of the cells of the animal includes a transgene. Other examples of transgenic animals include nonhuman primates, sheep, dogs, cows, goats, chickens, amphibians, etc. A transgene is exogenous DNA that is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a nonhuman animal, preferably a mammal, more preferably a mouse, in which an endogenous calpain protease gene has been altered by homologous recombination between the endogenous gene and an exogenous DNA molecule introduced into a cell of the animal, e.g., an embryonic cell of the animal, prior to development of the animal.

A transgenic animal of the invention can be created by introducing calpain protease-encoding nucleic acid into the male pronuclei of a fertilized oocyte, e.g., by microinjection, retroviral infection, and allowing the oocyte to develop in a pseudopregnant female foster animal. The calpain protease cDNA sequence can be introduced as a transgene into the genome of a nonhuman animal. Alternatively, a homologue of the mouse calpain protease gene can be isolated based on hybridization and used as a transgene. Intronic sequences and polyadenylation signals can also be included in the transgene to increase the efficiency of expression of the transgene. A tissue-specific regulatory sequence(s) can be operably linked to the calpain protease transgene to direct expression of calpain protease protein to particular cells. Methods for generating transgenic animals via embryo manipulation and microinjection, particularly animals such as mice, have become conventional in the art and are described, for example, in U.S. Pat. Nos. 4,736,866, 4,870,009, and 4,873,191 and in Hogan (1986) Manipulating the Mouse Embryo (Cold Spring Harbor Laboratory Press, Cold Spring Harbor, N.Y., 1986). Similar methods are used for production of other transgenic animals. A transgenic founder animal can be identified based upon the presence of the calpain protease transgene in its genome and/or expression of calpain protease mRNA in tissues or cells of the animals. A transgenic founder animal can then be used to breed additional animals carrying the transgene. Moreover, transgenic animals carrying a transgene encoding calpain protease gene can further be bred to other transgenic animals carrying other transgenes.

To create a homologous recombinant animal, one prepares a vector containing at least a portion of a calpain protease gene or a homolog of the gene into which a deletion, addition, or substitution has been introduced to thereby alter, e.g., functionally disrupt, the calpain protease gene. In a preferred embodiment, the vector is designed such that, upon homologous recombination, the endogenous calpain protease gene is functionally disrupted (i.e., no longer encodes a functional protein; also referred to as a “knock out” vector). Alternatively, the vector can be designed such that, upon homologous recombination, the endogenous calpain protease gene is mutated or otherwise altered but still encodes functional protein (e.g., the upstream regulatory region can be altered to thereby alter the expression of the endogenous calpain protease protein). In the homologous recombination vector, the altered portion of the calpain protease gene is flanked at its 5′ and 3′ ends by additional nucleic acid of the calpain protease gene to allow for homologous recombination to occur between the exogenous calpain protease gene carried by the vector and an endogenous calpain protease gene in an embryonic stem cell. The additional flanking calpain protease nucleic acid is of sufficient length for successful homologous recombination with the endogenous gene. Typically, several kilobases of flanking DNA (both at the 5′ and 3′ ends) are included in the vector (see, e.g., Thomas and Capecchi (1987) Cell 51:503 for a description of homologous recombination vectors). The vector is introduced into an embryonic stem cell line (e.g., by electroporation), and cells in which the introduced calpain protease gene has homologously recombined with the endogenous calpain protease gene are selected (see, e.g., Li et al. (1992) Cell 69:915). The selected cells are then injected into a blastocyst of an animal (e.g., a mouse) to form aggregation chimeras (see, e.g., Bradley (1987) in Teratocarcinomas and Embryonic Stem Cells: A Practical Approach, ed. Robertson (IRL, Oxford pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication Nos. WO 90/11354, WO 91/01140, WO 92/0968, and WO 93/04169.

In another embodiment, transgenic nonhuman animals containing selected systems that allow for regulated expression of the transgene can be produced. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355). If a crelloxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the nonhuman transgenic animals described herein can also be produced according to the methods described in Wilmut et al. (1997) Nature 385:810-813 and PCT Publication Nos. WO 97/07668 and WO 97/07669.

IV. Pharmaceutical Compositions

The calpain protease nucleic acid molecules, calpain protease proteins, and anti-calpain protease antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor ELθ (BASF; Parsippany, N.J.), or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride, in the composition. Prolonged absorption of the injectable compositions can be brought about by including in the composition an agent that delays absorption, for example, aluminum monostearate and gelatin.

Sterile injectable solutions can be prepared by incorporating the active compound (e.g., a calpain protease protein or anti-calpain protease antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying, which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth, or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the compounds are delivered in the form of an aerosol spray from a pressurized container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated with each unit containing a predetermined quantity of active compound calculated to produce the desired therapeutic effect in association with the required pharmaceutical carrier. Depending on the type and severity of the disease, about 1 μg/kg to about 15 mg/kg (e.g., 0.1 to 20 mg/kg) of antibody is an initial candidate dosage for administration to the patient, whether, for example, by one or more separate administrations, or by continuous infusion. A typical daily dosage might range from about 1 μg/kg to about 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays. An exemplary dosing regimen is disclosed in WO 94/04188. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (U.S. Pat. No. 5,328,470), or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, and antibodies described herein can be used in one or more of the following methods: (a) screening assays; (b) detection assays (e.g., chromosomal mapping, tissue typing, forensic biology); (c) predictive medicine (e.g., diagnostic assays, prognostic assays, monitoring clinical trials, and pharmacogenomics); and (d) methods of treatment (e.g., therapeutic and prophylactic). The isolated nucleic acid molecules of the invention can be used to express calpain protease protein (e.g., via a recombinant expression vector in a host cell in gene therapy applications), to detect calpain protease mRNA (e.g., in a biological sample) or a genetic lesion in a calpain protease gene, and to modulate calpain protease activity. In addition, the calpain protease proteins can be used to screen drugs or compounds that modulate the immune response as well as to treat disorders characterized by insufficient or excessive production of calpain protease protein or production of calpain protease protein forms that have decreased or aberrant activity compared to calpain protease wild type protein. In addition, the anti-calpain protease antibodies of the invention can be used to detect and isolate calpain protease proteins and modulate calpain protease activity.

The uses and methods of the invention apply particularly to the uses and methods in tissues in which expression of the calpain protease occurs in tissues including, but not limited to, normal tissue from colon, breast, lung, bone, ovary, spleen, kidney, heart, neuronal tissue, prostate, thymus, and T cells. Accordingly, the methods and uses apply particularly to these tissues and to disorders involving these tissues.

Disorders involving the spleen include, but are not limited to, splenomegaly, including nonspecific acute splenitis, congestive spenomegaly, and spenic infarcts; neoplasms, congenital anomalies, and rupture. Disorders associated with splenomegaly include infections, such as nonspecific splenitis, infectious mononucleosis, tuberculosis, typhoid fever, brucellosis, cytomegalovirus, syphilis, malaria, histoplasmosis, toxoplasmosis, kala-azar, trypanosomiasis, schistosomiasis, leishmaniasis, and echinococcosis; congestive states related to partial hypertension, such as cirrhosis of the liver, portal or splenic vein thrombosis, and cardiac failure; lymphohematogenous disorders, such as Hodgkin disease, non-Hodgkin lymphomas/leukemia, multiple myeloma, myeloproliferative disorders, hemolytic anemias, and thrombocytopenic purpura; immunologic-inflammatory conditions, such as rheumatoid arthritis and systemic lupus erythematosus; storage diseases such as Gaucher disease, Niemann-Pick disease, and mucopolysaccharidoses; and other conditions, such as amyloidosis, primary neoplasms and cysts, and secondary neoplasms.

Disorders involving the lung include, but are not limited to, congenital anomalies; atelectasis; diseases of vascular origin, such as pulmonary congestion and edema, including hemodynamic pulmonary edema and edema caused by microvascular injury, adult respiratory distress syndrome (diffuse alveolar damage), pulmonary embolism, hemorrhage, and infarction, and pulmonary hypertension and vascular sclerosis; chronic obstructive pulmonary disease, such as emphysema, chronic bronchitis, bronchial asthma, and bronchiectasis; diffuse interstitial (infiltrative, restrictive) diseases, such as pneumoconioses, sarcoidosis, idiopathic pulmonary fibrosis, desquamative interstitial pneumonitis, hypersensitivity pneumonitis, pulmonary eosinophilia (pulmonary infiltration with eosinophilia), Bronchiolitis obliterans-organizing pneumonia, diffuse pulmonary hemorrhage syndromes, including Goodpasture syndrome, idiopathic pulmonary hemosiderosis and other hemorrhagic syndromes, pulmonary involvement in collagen vascular disorders, and pulmonary alveolar proteinosis; complications of therapies, such as drug-induced lung disease, radiation-induced lung disease, and lung transplantation; tumors, such as bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma.

Disorders involving the colon include, but are not limited to, congenital anomalies, such as atresia and stenosis, Meckel diverticulum, congenital aganglionic megacolon-Hirschsprung disease; enterocolitis, such as diarrhea and dysentery, infectious enterocolitis, including viral gastroenteritis, bacterial enterocolitis, necrotizing enterocolitis, antibiotic-associated colitis (pseudomembranous colitis), and collagenous and lymphocytic colitis, miscellaneous intestinal inflammatory disorders, including parasites and protozoa, acquired immunodeficiency syndrome, transplantation, drug-induced intestinal injury, radiation enterocolitis, neutropenic colitis (typhlitis), and diversion colitis; idiopathic inflammatory bowel disease, such as Crohn disease and ulcerative colitis; tumors of the colon, such as non-neoplastic polyps, adenomas, familial syndromes, colorectal carcinogenesis, colorectal carcinoma, and carcinoid tumors.

Disorders involving T-cells include, but are not limited to, cell-mediated hypersensitivity, such as delayed type hypersensitivity and T-cell-mediated cytotoxicity, and transplant rejection; autoimmune diseases, such as systemic lupus erythematosus, Sjogren syndrome, systemic sclerosis, inflammatory myopathies, mixed connective tissue disease, and polyarteritis nodosa and other vasculitides; immunologic deficiency syndromes, including but not limited to, primary immunodeficiencies, such as thymic hypoplasia, severe combined immunodeficiency diseases, and AIDS; leukopenia; reactive (inflammatory) proliferations of white cells, including but not limited to, leukocytosis, acute nonspecific lymphadenitis, and chronic nonspecific lymphadenitis; neoplastic proliferations of white cells, including but not limited to lymphoid neoplasms, such as precursor T-cell neoplasms, such as acute lymphoblastic leukemia/lymphoma, peripheral T-cell and natural killer cell neoplasms that include peripheral T-cell lymphoma, unspecified, adult T-cell leukemia/lymphoma, mycosis fimgoides and Sezary syndrome, and Hodgkin disease.

Disorders involving the heart, include but are not limited to, heart failure, including but not limited to, cardiac hypertrophy, left-sided heart failure, and right-sided heart failure; ischemic heart disease, including but not limited to angina pectoris, myocardial infarction, chronic ischemic heart disease, and sudden cardiac death; hypertensive heart disease, including but not limited to, systemic (left-sided) hypertensive heart disease and pulmonary (right-sided) hypertensive heart disease; valvular heart disease, including but not limited to, valvular degeneration caused by calcification, such as calcific aortic stenosis, calcification of a congenitally bicuspid aortic valve, and mitral annular calcification, and myxomatous degeneration of the mitral valve (mitral valve prolapse), rheumatic fever and rheumatic heart disease, infective endocarditis, and noninfected vegetations, such as nonbacterial thrombotic endocarditis and endocarditis of systemic lupus erythematosus (Libman-Sacks disease), carcinoid heart disease, and complications of artificial valves; myocardial disease, including but not limited to dilated cardiomyopathy, hypertrophic cardiomyopathy, restrictive cardiomyopathy, and myocarditis; pericardial disease, including but not limited to, pericardial effusion and hemopericardium and pericarditis, including acute pericarditis and healed pericarditis, and rheumatoid heart disease; neoplastic heart disease, including but not limited to, primary cardiac tumors, such as myxoma, lipoma, papillary fibroelastoma, rhabdomyoma, and sarcoma, and cardiac effects of noncardiac neoplasms; congenital heart disease, including but not limited to, left-to-right shunts—late cyanosis, such as atrial septal defect, ventricular septal defect, patent ductus arteriosus, and atrioventricular septal defect, right-to-left shunts—early cyanosis, such as tetralogy of fallot, transposition of great arteries, truncus arteriosus, tricuspid atresia, and total anomalous pulmonary venous connection, obstructive congenital anomalies, such as coarctation of aorta, pulmonary stenosis and atresia, and aortic stenosis and atresia, and disorders involving cardiac transplantation.

Disorders involving the thymus include developmental disorders, such as DiGeorge syndrome with thymic hypoplasia or aplasia; thymic cysts; thymic hypoplasia, which involves the appearance of lymphoid follicles within the thymus, creating thymic follicular hyperplasia; and thymomas, including germ cell tumors, lymphomas, Hodgkin disease, and carcinoids. Thymomas can include benign or encapsulated thymoma, and malignant thymoma Type I (invasive thymoma) or Type II, designated thymic carcinoma.

Disorders involving the kidney include, but are not limited to, congenital anomalies including, but not limited to, cystic diseases of the kidney, that include but are not limited to, cystic renal dysplasia, autosomal dominant (adult) polycystic kidney disease, autosomal recessive (childhood) polycystic kidney disease, and cystic diseases of renal medulla, which include, but are not limited to, medullary sponge kidney, and nephronophthisis-uremic medullary cystic disease complex, acquired (dialysis-associated) cystic disease, such as simple cysts; glomerular diseases including pathologies of glomerular injury that include, but are not limited to, in situ immune complex deposition, that includes, but is not limited to, anti-GBM nephritis, Heymann nephritis, and antibodies against planted antigens, circulating immune complex nephritis, antibodies to glomerular cells, cell-mediated immunity in glomerulonephritis, activation of alternative complement pathway, epithelial cell injury, and pathologies involving mediators of glomerular injury including cellular and soluble mediators, acute glomerulonephritis, such as acute proliferative (poststreptococcal, postinfectious) glomerulonephritis, including but not limited to, poststreptococcal glomerulonephritis and nonstreptococcal acute glomerulonephritis, rapidly progressive (crescentic) glomerulonephritis, nephrotic syndrome, membranous glomeruloneplritis (membranous nephropathy), minimal change disease (lipoid nephrosis), focal segmental glomerulosclerosis, membranoproliferative glomerulonephritis, IgA nephropathy (Berger disease), focal proliferative and necrotizing glomerulonephritis (focal glomerulonephritis), hereditary nephritis, including but not limited to, Alport syndrome and thin membrane disease (benign familial hematuria), chronic glomerulonephritis, glomerular lesions associated with systemic disease, including but not limited to, systemic lupus erythematosus, Henoch-Schonlein purpura, bacterial endocarditis, diabetic glomerulosclerosis, amyloidosis, fibrillary and immunotactoid glomerulonephritis, and other systemic disorders; diseases affecting tubules and interstitium, including acute tubular necrosis and tubulointerstitial nephritis, including but not limited to, pyelonephritis and urinary tract infection, acute pyelonephritis, chronic pyelonephritis and reflux nephropathy, and tubulointerstitial nephritis induced by drugs and toxins, including but not limited to, acute drug-induced interstitial nephritis, analgesic abuse nephropathy, nephropathy associated with nonsteroidal anti-inflammatory drugs, and other tubulointerstitial diseases including, but not limited to, urate nephropathy, hypercalcemia and nephrocalcinosis, and multiple myeloma; diseases of blood vessels including benign nephrosclerosis, malignant hypertension and accelerated nephrosclerosis, renal artery stenosis, and thrombotic microangiopathies including, but not limited to, classic (childhood) hemolytic-uremic syndrome, adult hemolytic-uremic syndrome/thrombotic thrombocytopenic purpura, idiopathic HUS/TTP, and other vascular disorders including, but not limited to, atherosclerotic ischemic renal disease, atheroembolic renal disease, sickle cell disease nephropathy, diffuse cortical necrosis, and renal infarcts; urinary tract obstruction (obstructive uropathy); urolithiasis (renal calculi, stones); and tumors of the kidney including, but not limited to, benign tumors, such as renal papillary adenoma, renal fibroma or hamartoma (renomedullary interstitial cell tumor), angiomyolipoma, and oncocytoma, and malignant tumors, including renal cell carcinoma (hypemephroma, adenocarcinoma of kidney), which includes urothelial carcinomas of renal pelvis.

Disorders of the breast include, but are not limited to, disorders of development; inflammations, including but not limited to, acute mastitis, periductal mastitis, periductal mastitis (recurrent subareolar abscess, squamous metaplasia of lactiferous ducts), mammary duct ectasia, fat necrosis, granulomatous mastitis, and pathologies associated with silicone breast implants; fibrocystic changes; proliferative breast disease including, but not limited to, epithelial hyperplasia, sclerosing adenosis, and small duct papillomas; tumors including, but not limited to, stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, no special type, invasive lobular carcinoma, medullary carcinoma, colloid (mucinous) carcinoma, tubular carcinoma, and invasive papillary carcinoma, and miscellaneous malignant neoplasms.

Disorders in the male breast include, but are not limited to, gynecomastia and carcinoma.

Disorders involving the prostate include, but are not limited to, inflammations, benign enlargement, for example, nodular hyperplasia (benign prostatic hypertrophy or hyperplasia), and tumors such as carcinoma.

Disorders involving the thyroid include, but are not limited to, hyperthyroidism; hypothyroidism including, but not limited to, cretinism and myxedema; thyroiditis including, but not limited to, hashimoto thyroiditis, subacute (granulomatous) thyroiditis, and subacute lymphocytic (painless) thyroiditis; Graves disease; diffuse and multinodular goiter including, but not limited to, diffuse nontoxic (simple) goiter and multinodular goiter; neoplasms of the thyroid including, but not limited to, adenomas, other benign tumors, and carcinomas, which include, but are not limited to, papillary carcinoma, follicular carcinoma, medullary carcinoma, and anaplastic carcinoma; and cogenital anomalies.

Disorders involving precursor T-cell neoplasms include precursor T lymphoblastic leukemia/lymphoma. Disorders involving peripheral T-cell and natural killer cell neoplasms include T-cell chronic lymphocytic leukemia, large granular lymphocytic leukemia, mycosis fungoides and Sezary syndrome, peripheral T-cell lymphoma, unspecified, angioimmunoblastic T-cell lymphoma, angiocentric lymphoma (NK/T-cell lymphoma^{4a}), intestinal T-cell lymphoma, adult T-cell leukemia/lymphoma, and anaplastic large cell lymphoma.

Preferred disorders include carcinoma of the breast and colon. Further disorders to which the uses and methods of the present invention particularly pertain include lung carcinoma. Uses and methods also apply to tumors involving the parythyroid.

The gene has been mapped to chromosome 3 p21-24. Nearby mutations/loci include human-SCCL, small cell cancer of the lung; pancreatic endocrine tumor suppressor 1; CMD1E; cardiomyopathy, dilated 1E; DFNB6, deafness, neurosensory, autosomal recessive 6; Moyamoya disease; FANCD, Fanconi anemia, complementation group D; pancreatic endocrine tumor suppressor 1; Marfan-like connective tissue disorder; SCCL, small cell cancer of the lung; progressive external ophthalmoplegia, TYPE 2; LRS1 Larsen syndrome, autosomal dominant; RCC1, renal cell carcinoma 1; Mouse-Mouse-Sluc3, susceptibility to lung cancer 3; Ots1, ovarian teratoma susceptibility 1; Cor, distribution of corticosterone in adrenal cortex cells; cdf, cerebellar deficient folia; mnd2, motor neuron degeneration 2; tc, truncate; fe, faded; Cia3, collagen induced arthritis QTL 3; Ldr2, lactate dehydrogenase regulator 2; Cyx, cycloheximide tasting; Qui, quinine sensitivity, taste; Cd, crooked; Rua, raffinose acetate tasting: Nearby known genes include, but are not limited to, BTD, SAB, KIAA0210, SATB1, SEMA3F, RAB5A, PCAF, UBE2E1, NR1D2, RPL15, RARB, TOP2B, THRB, TDGF1, TGFBR2, CTNNB1, MLH1.

RCC1 has a number of mutated genes associated with the locus. Predisposition to renal cancer in one family has been associated with an inherited chromosomal translocation, t(3:8) (p21:q24) (Cohen et al. (1979) New Eng. J. Med. 301:592-595). It was further demonstrated that in one patient, the breakpoints occurred at sub bands 3p14.2 (not 3p21) and 8q24.1 (Cancer Genet. Cytogenet. 11:479-481 (1984)). The 3p14.2 region also contains FRA3B, the most sensitive fragile site induced by aphidicolin. A gene referred to as HRCA1 (hereditary renal cancer-associated 1) was identified as mapping immediately adjacent to the breakpoint. On the basis of the chromosomal position, it was considered to be a candidate tumor suppressor gene (Boldog et al., Proc. Nat. Acad. Sci. 90:8509-8513 (1993)).

The SCCL locus has been associated with a deletion in the 3p region (Whang-Peng et al. (1982) Science 215:181-182). The deletion was specifically mapped to 3p (14-23). Using a molecular genetic approach, Kok et al. (Nature 330: 578-581 (1987)) found evidence for consistent deletion at the 3p21 region not only in SCCL but in all major types of lung cancer. Johnson et al. (J. Clin. Invest. 82:502-507 (1988)) found the homozygous loss of at least one marker in the region 3p14-p21 in tumor tissue of 23 out of 25 patients. Accordingly, three molecular mechanisms have been proposed to be involved in the development of lung cancer: deletion of 3p, deregulated expression of the MYC family of genes and growth factors and a constitutive 3p14.2 fragile site (Birrer et al., Semin. Oncol. 15:226-235 (1988)).

Accordingly, further disorders to which the calpain protease is relevant include small cell cancer of the lung and renal cell carcinoma.

With respect to the genes and loci in the corresponding region of the mouse genome, SLUC3, QTS, and COR are of particular relevance. SLUC3 influences the susceptibility to lung cancer in the mouse (Fijneman et al., Nat. Genet. 14:465-467 (1996)).

A. Screening Assays

The invention provides a method (also referred to herein as a “screening assay”) for identifying modulators, i.e., candidate or test compounds or agents (e.g., peptides, peptidomimetics, small molecules, or other drugs) that bind to calpain protease proteins or have a stimulatory or inhibitory effect on, for example, calpain protease expression or calpain protease activity.

The test compounds of the present invention can be obtained using any of the numerous approaches in combinatorial library methods known in the art, including biological libraries, spatially addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, nonpeptide oligomer, or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869), orphage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310).

Determining the ability of the test compound to bind to the calpain protease protein can be accomplished, for example, by coupling the test compound with a radioisotope or enzymatic label such that binding of the test compound to the calpain protease protein or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioeemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

In a similar manner, one may determine the ability of the calpain protease protein to bind to or interact with a calpain protease target molecule. By “target molecule” is intended a molecule with which a calpain protease protein binds or interacts in nature. In a preferred embodiment, the ability of the calpain protease protein to bind to or interact with a calpain protease target molecule can be determined by monitoring the activity of the target molecule. For example, the activity of the target molecule can be monitored by detecting induction of a cellular second messenger of the target (e.g., intracellular Ca²⁺, diacylglycerol, IP3, etc.), detecting catalytic/enzymatic activity of the target on an appropriate substrate, detecting the induction of a reporter gene (e.g., a calpain protease-responsive regulatory element operably linked to a nucleic acid encoding a detectable marker, e.g. luciferase), or detecting a cellular response, for example, cellular differentiation or cell proliferation.

In yet another embodiment, an assay of the present invention is a cell-free assay comprising contacting a calpain protease protein or biologically active portion thereof with a test compound and determining the ability of the test compound to bind to the calpain protease protein or biologically active portion thereof. Binding of the test compound to the calpain protease protein can be determined either directly or indirectly as described above. hi a preferred embodiment, the assay includes contacting the calpain protease protein or biologically active portion thereof with a known compound that binds calpain protease protein to form an assay mixture, contacting the assay mixture with a test compound, and determining the ability of the test compound to preferentially bind to calpain protease protein or biologically active portion thereof as compared to the known compound.

In another embodiment, an assay is a cell-free assay comprising contacting calpain protease protein or biologically active portion thereof with a test compound and determining the ability of the test compound to modulate (e.g., stimulate or inhibit) the activity of the calpain protease protein or biologically active portion thereof. Determining the ability of the test compound to modulate the activity of a calpain protease protein can be accomplished, for example, by determining the ability of the calpain protease protein to bind to a calpain protease target molecule as described above for determining direct binding. In an alternative embodiment, determining the ability of the test compound to modulate the activity of a calpain protease protein can be accomplished by determining the ability of the calpain protease protein to further modulate a calpain protease target molecule. For example, the catalytic/enzymatic activity of the target molecule on an appropriate substrate can be determined as previously described.

In yet another embodiment, the cell-free assay comprises contacting the calpain protease protein or biologically active portion thereof with a known compound that binds a calpain protease protein to form an assay mixture, contacting the assay mixture with a test compound, and determining the ability of the test compound to preferentially bind to or modulate the activity of a calpain protease target molecule.

In the above-mentioned assays, it may be desirable to immobilize either a calpain protease protein or its target molecule to facilitate separation of complexed from uncomplexed forms of one or both of the proteins, as well as to accommodate automation of the assay. In one embodiment, a fusion protein can be provided that adds a domain that allows one or both of the proteins to be bound to a matrix. For example, glutathione-S-transferase/calpain protease fusion proteins or glutathione-S-transferase/target fusion proteins can be adsorbed onto glutathione sepharose beads (Sigma Chemical, St. Louis, Mo.) or glutathione-derivatized microtitre plates, which are then combined with the test compound or the test compound and either the nonabsorbed target protein or calpain protease protein, and the mixture incubated under conditions conducive to complex formation (e.g., at physiological conditions for salt and pH). Following incubation, the beads or microtitre plate wells are washed to remove any unbound components and complex formation is measured either directly or indirectly, for example, as described above. Alternatively, the complexes can be dissociated from the matrix, and the level of calpain protease binding or activity determined using standard techniques.

Other techniques for immobilizing proteins on matrices can also be used in the screening assays of the invention. For example, either calpain protease protein or its target molecule can be immobilized utilizing conjugation of biotin and streptavidin. Biotinylated calpain protease molecules or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96-well plates (Pierce Chemicals). Alternatively, antibodies reactive with a calpain protease protein or target molecules but which do not interfere with binding of the calpain protease protein to its target molecule can be derivatized to the wells of the plate, and unbound target or calpain protease protein trapped in the wells by antibody conjugation. Methods for detecting such complexes, in addition to those described above for the GST-immobilized complexes, include immunodetection of complexes using antibodies reactive with the calpain protease protein or target molecule, as well as enzyme-linked assays that rely on detecting an enzymatic activity associated with the calpain protease protein or target molecule.

In another embodiment, modulators of calpain protease expression are identified in a method in which a cell is contacted with a candidate compound and the expression of calpain protease mRNA or protein in the cell is determined relative to expression of calpain protease mRNA or protein in a cell in the absence of the candidate compound. When expression is greater (statistically significantly greater) in the presence of the candidate compound than in its absence, the candidate compound is identified as a stimulator of calpain protease mRNA or protein expression. Alternatively, when expression is less (statistically significantly less) in the presence of the candidate compound than in its absence, the candidate compound is identified as an inhibitor of calpain protease mRNA or protein expression. The level of calpain protease mRNA or protein expression in the cells can be determined by methods described herein for detecting calpain protease mRNA or protein.

In yet another aspect of the invention, the calpain protease proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300), to identify other proteins, which bind to or interact with calpain protease protein (“calpain protease-binding proteins” or “calpain protease-bp”) and modulate calpain protease activity. Such calpain protease-binding proteins are also likely to be involved in the propagation of signals by the calpain protease proteins as, for example, upstream or downstream elements of the calpain protease pathway.

This invention further pertains to novel agents identified by the above-described screening assays and uses thereof for treatments as described herein.

B. Detection Assays

Portions or fragments of the cDNA sequences identified herein (and the corresponding complete gene sequences) can be used in numerous ways as polynucleotide reagents. For example, these sequences can be used to: (1) map their respective genes on a chromosome; (2) identify an individual from a minute biological sample (tissue typing); and (3) aid in forensic identification of a biological sample. These applications are described in the subsections below.

1. Chromosome Mapping

The isolated complete or partial calpain protease gene sequences of the invention can be used to map their respective calpain protease genes on a chromosome, thereby facilitating the location of gene regions associated with genetic disease. Computer analysis of calpain protease sequences can be used to rapidly select PCR primers (preferably 15-25 bp in length) that do not span more than one exon in the genomic DNA, thereby simplifying the amplification process. These primers can then be used for PCR screening of somatic cell hybrids containing individual human chromosomes. Only those hybrids containing the human gene corresponding to the calpain protease sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow (because they lack a particular enzyme), but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes (D'Eustachio et al. (1983) Science 220:919-924). Somatic cell hybrids containing only fragments of human chromosomes can also be produced by using human chromosomes with translocations and deletions.

Other mapping strategies that can similarly be used to map a calpain protease sequence to its chromosome include in situ hybridization (described in Fan et al. (1990) Proc. Natl. Acad. Sci. USA 87:6223-27), pre-screening with labeled flow-sorted chromosomes, and pre-selection by hybridization to chromosome specific cDNA libraries. Furthermore, fluorescence in situ hybridization (FISH) of a DNA sequence to a metaphase chromosomal spread can be used to provide a precise chromosomal location in one step. For a review of this technique, see Verma eta a. (1988) Human Chromosomes: A Manual ofBasic Techniques (Pergamon Press, N.Y.). The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results in a reasonable amount of time.

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland et al. (1987) Nature 325:783-787.

Moreover, differences in the DNA sequences between individuals affected and unaffected with a disease associated with the calpain protease gene can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The calpain protease sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes and probed on a Southern blot to yield unique bands for identification. The sequences of the present invention are useful as additional DNA markers for RFLP (described in U.S. Pat. No. 5,272,057).

Furthermore, the sequences of the present invention can be used to provide an alternative technique for determining the actual base-by-base DNA sequence of selected portions of an individual's genome. Thus, the calpain protease sequences of the invention can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The calpain protease sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. The noncoding sequences of SEQ ID NO:1 can comfortably provide positive individual identification with a panel of perhaps 10 to 1,000 primers that each yield a noncoding amplified sequence of 100 bases. If a predicted coding sequence, such as that in SEQ ID NO:1, is used, a more appropriate number of primers for positive individual identification would be 500 to 2,000.

3. Use of Partial Calpain Protease Sequences in Forensic Biology

DNA-based identification techniques can also be used in forensic biology. In this manner, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” that is unique to a particular individual. As mentioned above, actual base sequence information can be used for identification as an accurate alternative to patterns formed by restriction enzyme generated fragments. Sequences targeted to noncoding regions of SEQ ID NO:1 are particularly appropriate for this use as greater numbers of polymorphisms occur in the noncoding regions, making it easier to differentiate individuals using this technique. Examples of polynucleotide reagents include the calpain protease sequences or portions thereof, e.g., fragments derived from the noncoding regions of SEQ ID NO:1 having a length of at least 20 or 30 bases.

The calpain protease sequences described herein can further be used to provide polynucleotide reagents, e.g., labeled or labelable probes that can be used in, for example, an in situ hybridization technique, to identify a specific tissue. This can be very useful in cases where a forensic pathologist is presented with a tissue of unknown origin. Panels of such calpain protease probes, can be used to identify tissue by species and/or by organ type.

In a similar fashion, these reagents, e.g., calpain protease primers or probes can be used to screen tissue culture for contamination (i.e., screen for the presence of a mixture of different types of cells in a culture).

C. Predictive Medicine

The present invention also pertains to the field of predictive medicine in which diagnostic assays, prognostic assays, pharmacogenomics, and monitoring clinical trails are used for prognostic (predictive) purposes to thereby treat an individual prophylactically. These applications are described in the subsections below.

1. Diagnostic Assays

One aspect of the present invention relates to diagnostic assays for detecting calpain protease protein and/or nucleic acid expression as well as calpain protease activity, in the context of a biological sample. An exemplary method for detecting the presence or absence of calpain protease proteins in a biological sample involves obtaining a biological sample from a test subject and contacting the biological sample with a compound or an agent capable of detecting calpain protease protein or nucleic acid (e.g., mRNA, genomic DNA) that encodes calpain protease protein such that the presence of calpain protease protein is detected in the biological sample. Results obtained with a biological sample from the test subject may be compared to results obtained with a biological sample from a control subject.

A preferred agent for detecting calpain protease mRNA or genomic DNA is a labeled nucleic acid probe capable of hybridizing to calpain protease mRNA or genomic DNA. The nucleic acid probe can be, for example, a full-length calpain protease nucleic acid, such as the nucleic acid of SEQ ID NO:1, or a portion thereof, such as a nucleic acid molecule of at least 15, 30, 50, 100, 250, or 500 nucleotides in length and sufficient to specifically hybridize under stringent conditions to calpain protease mRNA or genomic DNA. Other suitable probes for use in the diagnostic assays of the invention are described herein.

A preferred agent for detecting calpain protease protein is an antibody capable of binding to calpain protease protein, preferably an antibody with a detectable label. Antibodies can be polyclonal, or more preferably, monoclonal. An intact antibody, or a fragment thereof (e.g., Fab or F(abN)₂)can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The term “biological sample” is intended to include tissues, cells, and biological fluids isolated from a subject, as well as tissues, cells, and fluids present within a subject. That is, the detection method of the invention can be used to detect calpain protease mRNA, protein, or genomic DNA in a biological sample in vitro as well as in vivo. For example, in vitro techniques for detection of calpain protease mRNA include Northern hybridizations and in situ hybridizations. In vitro techniques for detection of calpain protease protein include enzyme linked immunosorbent assays (ELISAs), Western blots, immunoprecipitations, and immunofluorescence. In vitro techniques for detection of calpain protease genomic DNA include Southern hybridizations. Furthermore, in vivo techniques for detection of calpain protease protein include introducing into a subject a labeled anti-calpain protease antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

The invention also encompasses kits for detecting the presence of calpain protease proteins in a biological sample (a test sample). Such kits can be used to determine if a subject is suffering from or is at increased risk of developing a disorder associated with aberrant expression of calpain protease protein (e.g., an immunological disorder). For example, the kit can comprise a labeled compound or agent capable of detecting calpain protease protein or mRNA in a biological sample and means for determining the amount of a calpain protease protein in the sample (e.g., an anti-calpain protease antibody or an oligonucleotide probe that binds to DNA encoding a calpain protease protein, e.g., SEQ ID NO:1). Kits can also include instructions for observing that the tested subject is suffering from or is at risk of developing a disorder associated with aberrant expression of calpain protease sequences if the amount of calpain protease protein or mRNA is above or below a normal level.

For antibody-based kits, the kit can comprise, for example: (1) a first antibody (e.g., attached to a solid support) that binds to calpain protease protein; and, optionally, (2) a second, different antibody that binds to calpain protease protein or the first antibody and is conjugated to a detectable agent. For oligonucleotide-based kits, the kit can comprise, for example: (1) an oligonucleotide, e.g., a detectably labeled oligonucleotide, that hybridizes to a calpain protease nucleic acid sequence or (2) a pair of primers useful for amplifying a calpain protease nucleic acid molecule.

The kit can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples that can be assayed and compared to the test sample contained. Each component of the kit is usually enclosed within an individual container, and all of the various containers are within a single package along with instructions for observing whether the tested subject is suffering from or is at risk of developing a disorder associated with aberrant expression of calpain protease proteins.

2. Prognostic Assays

The methods described herein can furthermore be utilized as diagnostic or prognostic assays to identify subjects having or at risk of developing a disease or disorder associated with calpain protease protein, calpain protease nucleic acid expression, or calpain protease activity. Prognostic assays can be used for prognostic or predictive purposes to thereby prophylactically treat an individual prior to the onset of a disorder characterized by or associated with calpain protease protein, calpain protease nucleic acid expression, or calpain protease activity.

Thus, the present invention provides a method in which a test sample is obtained from a subject, and calpain protease protein or nucleic acid (e.g., mRNA, genomic DNA) is detected, wherein the presence of calpain protease protein or nucleic acid is diagnostic for a subject having or at risk of developing a disease or disorder associated with aberrant calpain protease expression or activity. As used herein, a “test sample” refers to a biological sample obtained from a subject of interest. For example, a test sample can be a biological fluid (e.g., serum), cell sample, or tissue.

Furthermore, using the prognostic assays described herein, the present invention provides methods for determining whether a subject can be administered a specific agent (e.g., an agonist, antagonist, peptidomimetic, protein, peptide, nucleic acid, small molecule, or other drug candidate) or class of agents (e.g., agents of a type that decrease calpain protease activity) to effectively treat a disease or disorder associated with aberrant calpain protease expression or activity. In this manner, a test sample is obtained and calpain protease protein or nucleic acid is detected. The presence of calpain protease protein or nucleic acid is diagnostic for a subject that can be administered the agent to treat a disorder associated with aberrant calpain protease expression or activity.

The methods of the invention can also be used to detect genetic lesions or mutations in a calpain protease gene, thereby determining if a subject with the lesioned gene is at risk for a disorder characterized by aberrant cell proliferation and/or differentiation. In preferred embodiments, the methods include detecting, in a sample of cells from the subject, the presence or absence of a genetic lesion or mutation characterized by at least one of an alteration affecting the integrity of a gene encoding a calpain protease protein, or the misexpression of the calpain protease gene. For example, such genetic lesions or mutations can be detected by ascertaining the existence of at least one of: (1) a deletion of one or more nucleotides from a calpain protease gene; (2) an addition of one or more nucleotides to a calpain protease gene; (3) a substitution of one or more nucleotides of a calpain protease gene; (4) a chromosomal rearrangement of a calpain protease gene; (5) an alteration in the level of a messenger RNA transcript of a calpain protease gene; (6) an aberrant modification of a calpain protease gene, such as of the methylation pattern of the genomic DNA; (7) the presence of a non-wild-type splicing pattern of a messenger RNA transcript of a calpain protease gene; (8) a non-wild-type level of a calpain protease-protein; (9) an allelic loss of a calpain protease gene; and (10) an inappropriate post-translational modification of a calpain protease protein. As described herein, there are a large number of assay techniques known in the art that can be used for detecting lesions in a calpain protease gene. Any cell type or tissue, preferably peripheral blood leukocytes, in which calpain protease proteins are expressed may be utilized in the prognostic assays described herein.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the calpain protease-gene (see, e.g., Abravaya et al. (1995) Nucleic Acids Res. 23:675-682). It is anticipated that PCR and/or LCR may be desirable to use as a preliminary amplification step in conjunction with any of the techniques used for detecting mutations described herein.

Alternative amplification methods include self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a calpain protease gene from a sample cell can be identified by alterations in restriction enzyme cleavage patterns of isolated test sample and control DNA digested with one or more restriction endonucleases. Moreover, the use of sequence specific ribozymes (see, e.g., U.S. Pat. No. 5,498,531) can be used to score for the presence of specific mutations by development or loss of a ribozyme cleavage site.

In other embodiments, genetic mutations in a calpain protease molecule can be identified by hybridizing a sample and control nucleic acids, e.g., DNA or RNA, to high density arrays containing hundreds or thousands of oligonucleotides probes (Cronin et al. (1996) Human Mutation 7:244-255; Kozal et al. (1996) Nature Medicine 2:753-759). In yet another embodiment, any of a variety of sequencing reactions known in the art can be used to directly sequence the calpain protease gene and detect mutations by comparing the sequence of the sample calpain protease gene with the corresponding wild-type (control) sequence. Examples of sequencing reactions include those based on techniques developed by Maxim and Gilbert ((1977) Proc. Natl. Acad. Sci. USA 74:560) or Sanger ((1977) Proc. Natl.. Acad. Sci. USA 74:5463). It is also contemplated that any of a variety of automated sequencing procedures can be utilized when performing the diagnostic assays ((1995) Bio/Techniques 19:448), including sequencing by mass spectrometry (see, e.g., PCT Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the calpain protease gene include methods in which protection from cleavage agents is used to detect mismatched bases in RNA/RNA or RNA/DNA heteroduplexes (Myers et al. (1985) Science 230:1242). See, also Cotton et al. (1988) Proc. Natl. Acad. Sci. USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more “DNA mismatch repair” enzymes that recognize mismatched base pairs in double-stranded DNA in defined systems for detecting and mapping point mutations in calpain protease cDNAs obtained from samples of cells. See, e.g., Hsu et al. (1994) Carcinogenesis 15:1657-1662. According to an exemplary embodiment, a probe based on a calpain protease sequence, e.g., a wild-type calpain protease sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in calpain protease genes. For example, single-strand conformation polymorphism (SSCP) may be used to detect differences in electrophoretic mobility between mutant and wild-type nucleic acids (Orita et al. (1989) Proc. Natl. Acad. Sci. USA 86:2766; see also Cotton (1993) Mutat. Res. 285:125-144; Hayashi (1992) Genet. Anal. Tech. AppL. 9:73-79). The sensitivity of the assay may be enhanced by using RNA (rather than DNA), in which the secondary structure is more sensitive to a change in sequence. In a preferred embodiment, the subject method utilizes heteroduplex analysis to separate double-stranded heteroduplex molecules on the basis of changes in electrophoretic mobility (Keen et al. (1991) Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found (Saiki et al. (1986) Nature 324:163); Saiki et al. (1989) Proc. Natl. Acad. Sci. USA 86:6230). Such allele-specific oligonucleotides are hybridized to PCR-amplified target DNA or a number of different mutations when the oligonucleotides are attached to the hybridizing membrane and hybridized with labeled target DNA.

Alternatively, allele-specific amplification technology, which depends on selective PCR amplification, may be used in conjunction with the instant invention. Oligonucleotides used as primers for specific amplification may carry the mutation of interest in the center of the molecule so that amplification depends on differential hybridization (Gibbs et al. (1989) Nucleic Acids Res. 17:2437-2448) or at the extreme 3N end of one primer where, under appropriate conditions, mismatch can prevent or reduce polymerase extension (Prossner (1993) Tibtech 11:238). In addition, it maybe desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl.. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing prepackaged diagnostic kits comprising at least one probe nucleic acid or antibody reagent described herein, which may be conveniently used, e.g., in clinical settings to a diagnosed patients exhibiting symptoms or family history of a disease or illness involving a calpain protease gene.

3. Pharmacogenomics

Agents, or modulators that have a stimulatory or inhibitory effect on calpain protease activity (e.g., calpain protease gene expression) as identified by a screening assay described herein, can be administered to individuals to treat (prophylactically or therapeutically) disorders associated with aberrant calpain protease activity as well as to modulate the phenotype of an immune response. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of calpain protease protein, expression of calpain protease nucleic acid, or mutation content of calpain protease genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms. For example, glucose-6-phosphate dehydrogenase deficiency (G6PD) is a common inherited enzymopathy in which the main clinical complication is haemolysis after ingestion of oxidant drugs (antimalarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of calpain protease protein, expression of calpain protease nucleic acid, or mutation content of calpain protease genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a calpain protease modulator, such as a modulator identified by one of the exemplary screening assays described herein.

4. Monitoring of Effects During Clinical Trials

Monitoring the influence of agents (e.g., drugs, compounds) on the expression or activity of calpain protease genes (e.g., the ability to modulate aberrant cell proliferation and/or differentiation) can be applied not only in basic drug screening but also in clinical trials. For example, the effectiveness of an agent, as determined by a screening assay as described herein, to increase or decrease calpain protease gene expression, protein levels, or protein activity, can be monitored in clinical trials of subjects exhibiting decreased or increased calpain protease gene expression, protein levels, or protein activity. In such clinical trials, calpain protease expression or activity and preferably that of other genes that have been implicated in for example, a cellular proliferation disorder, can be used as a marker of the immune responsiveness of a particular cell.

For example, and not by way of limitation, genes that are modulated in cells by treatment with an agent (e.g., compound, drug, or small molecule) that modulates calpain protease activity (e.g., as identified in a screening assay described herein) can be identified. Thus, to study the effect of agents on cellular proliferation disorders, for example, in a clinical trial, cells can be isolated and RNA prepared and analyzed for the levels of expression of calpain protease genes and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of calpain protease genes or other genes. In this way, the gene expression pattern can serve as a marker, indicative of the physiological response of the cells to the agent. Accordingly, this response state may be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (1) obtaining a preadministration sample from a subject prior to administration of the agent; (2) detecting the level of expression of a calpain protease protein, mRNA, or genomic DNA in the preadministration sample; (3) obtaining one or more postadministration samples from the subject; (4) detecting the level of expression or activity of the calpain protease protein, mRNA, or genomic DNA in the postadministration samples; (5) comparing the level of expression or activity of the calpain protease protein, mRNA, or genomic DNA in the preadministration sample with the calpain protease protein, mRNA, or genomic DNA in the postadministration sample or samples; and (vi) altering the administration of the agent to the subject accordingly to bring about the desired effect, i.e., for example, an increase or a decrease in the expression or activity of a calpain protease protein.

C. Methods of Treatment

The present invention provides for both prophylactic and therapeutic methods of treating a subject at risk of (or susceptible to) a disorder or having a disorder associated with aberrant calpain protease expression or activity. Additionally, the compositions of the invention find use in the treatment of disorders described herein. Thus, therapies for disorders associated with altered calpain protease activity are encompassed. Such disorders include, but are not limited to, disorders associated with perturbed cellular growth and differentiation; exercise-induced injury and repair; apoptosis including T-cell receptor-induced apoptosis, HIV-infected cell apoptosis, ectoposide-treated cell apoptosis, nerve growth factor deprived neuronal apoptosis; ischemia; traumatic brain injury; Alzheimer's disease and other neurodegenerative diseases; demyelinating diseases including experimental allergic encephalomyelitis (EAE) and multiple sclerosis; LGMD2A muscular dystrophy; spinal cord injury (SCI); proliferative disorders or differentiative disorders such as cancer, e.g., melanoma, prostate cancer, cervical cancer, breast cancer, colon cancer, or sarcoma; and renal cell death associated with diverse toxicants.

Further, as discussed in the exemplary section herein, the expression of the calpain protease has been identified in specific tissues and accordingly is related to disorders involving these tissues. Thus, methods of treatment extend to such disorders and tissues.

1. Prophylactic Methods

In one aspect, the invention provides a method for preventing in a subject a disease or condition associated with an aberrant calpain protease expression or activity by administering to the subject an agent that modulates calpain protease expression or at least one calpain protease gene activity. Subjects at risk for a disease that is caused, or contributed to, by aberrant calpain protease expression or activity can be identified by, for example, any or a combination of diagnostic or prognostic assays as described herein. Administration of a prophylactic agent can occur prior to the manifestation of symptoms characteristic of the calpain protease aberrancy, such that a disease or disorder is prevented or, alternatively, delayed in its progression. Depending on the type of calpain protease aberrancy, for example, a calpain protease agonist or calpain protease antagonist agent can be used for treating the subject. The appropriate agent can be determined based on screening assays described herein.

2. Therapeutic Methods

Another aspect of the invention pertains to methods of modulating calpain protease expression or activity for therapeutic purposes. The modulatory method of the invention involves contacting a cell with an agent that modulates one or more of the activities of calpain protease protein activity associated with the cell. An agent that modulates calpain protease protein activity can be an agent as described herein, such as a nucleic acid or a protein, a naturally-occurring cognate ligand of a calpain protease protein, a peptide, a calpain protease peptidomimetic, or other small molecule. In one embodiment, the agent stimulates one or more of the biological activities of calpain protease protein. Examples of such stimulatory agents include active calpain protease protein and a nucleic acid molecule encoding a calpain protease protein that has been introduced into the cell. In another embodiment, the agent inhibits one or more of the biological activities of calpain protease protein. Examples of such inhibitory agents include antisense calpain protease nucleic acid molecules and anti-calpain protease antibodies.

These modulatory methods can be performed in vitro (e.g., by culturing the cell with the agent) or, alternatively, in vivo (e.g, by administering the agent to a subject). As such, the present invention provides methods of treating an individual afflicted with a disease or disorder characterized by aberrant expression or activity of a calpain protease protein or nucleic acid molecule. In one embodiment, the method involves administering an agent (e.g., an agent identified by a screening assay described herein), or a combination of agents, that modulates (e.g., upregulates or downregulates) calpain protease expression or activity. In another embodiment, the method involves administering a calpain protease protein or nucleic acid molecule as therapy to compensate for reduced or aberrant calpain protease expression or activity.

Stimulation of calpain protease activity is desirable in situations in which a calpain protease protein is abnormally downregulated and/or in which increased calpain protease activity is likely to have a beneficial effect. Conversely, inhibition of calpain protease activity is desirable in situations in which calpain protease activity is abnormally upregulated and/or in which decreased calpain protease activity is likely to have a beneficial effect.

This invention is further illustrated by the following examples, which should not be construed as limiting.

### EXPERIMENTAL

### EXAMPLE 1

### Isolation of h26176

Clone h26176 was isolated from a human T-cell cDNA library. The identified clone h26176 encodes a transcript of approximately 3.78 Kb (corresponding cDNA set forth in SEQ ID NO:1). The open reading frame (nucleotides 276-2714) of this transcript encodes a predicted 813 amino acid protein (SEQ ID NO:2)

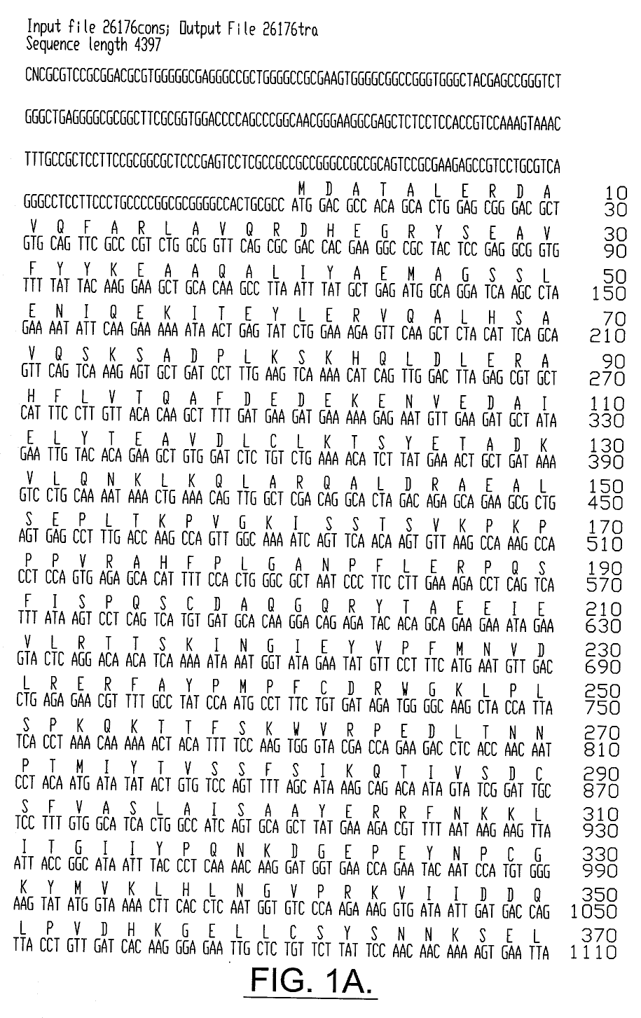

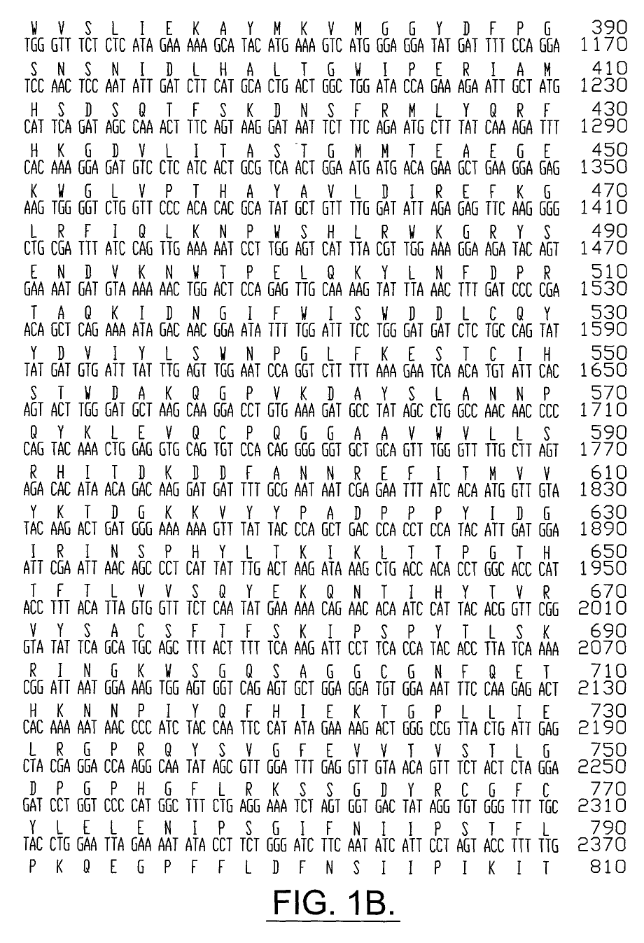

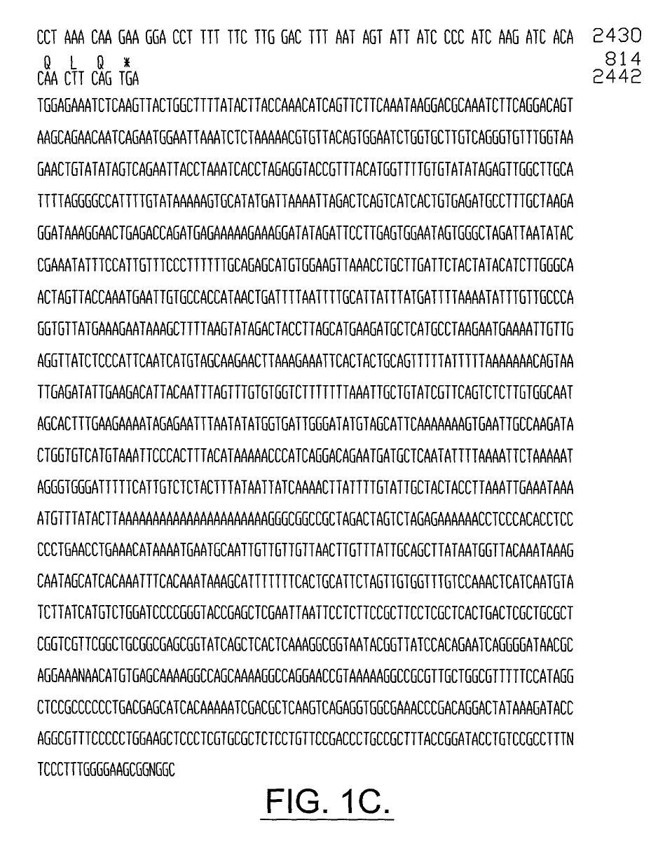

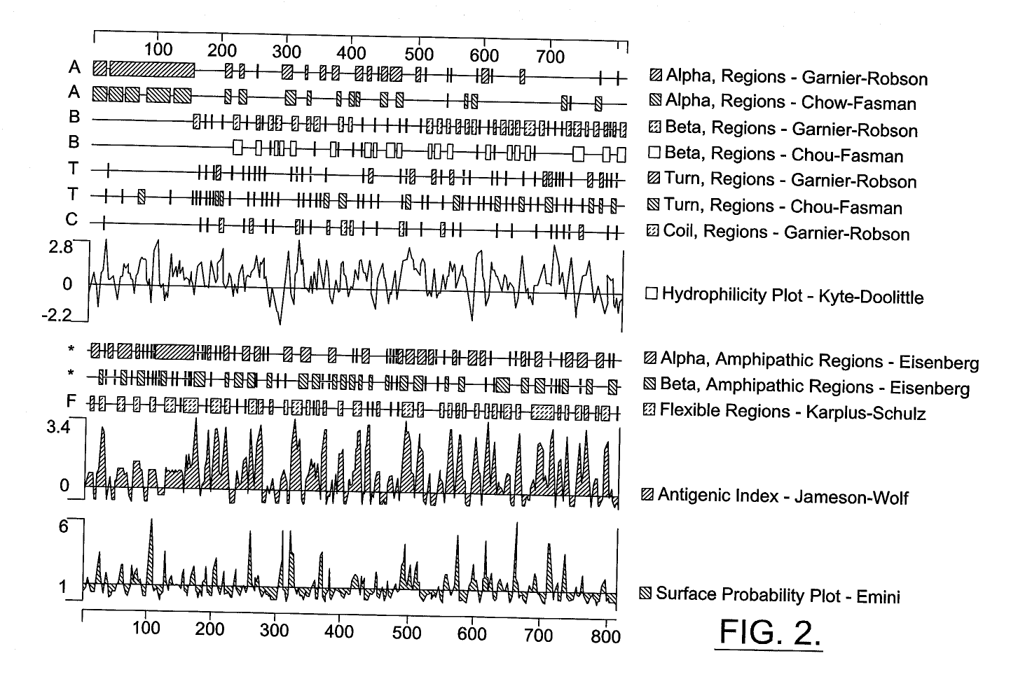

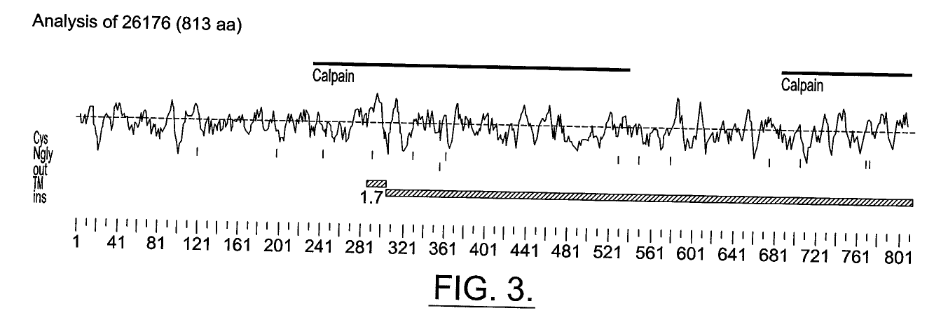

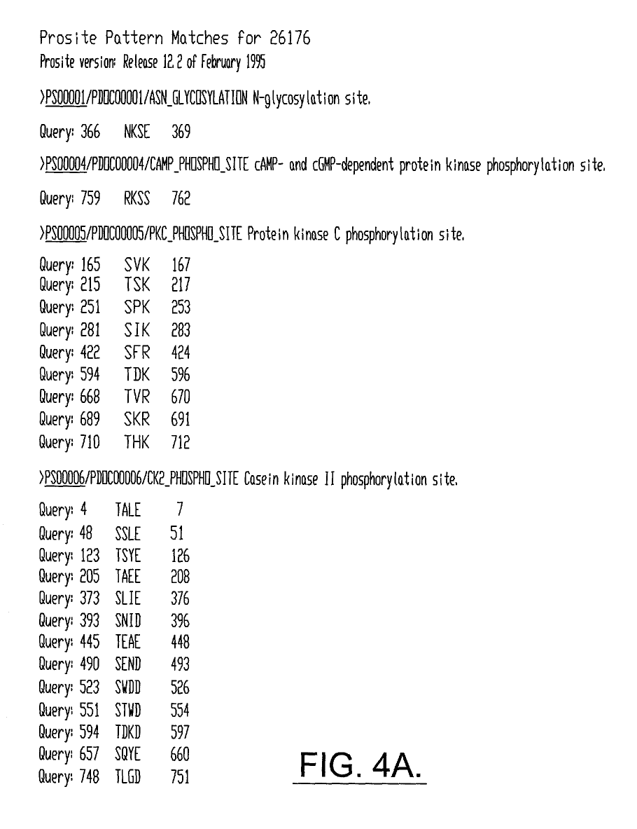

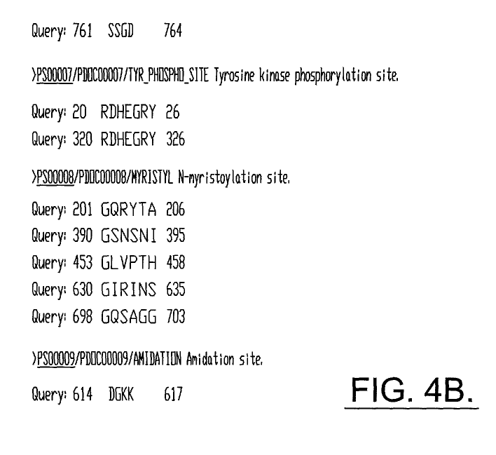

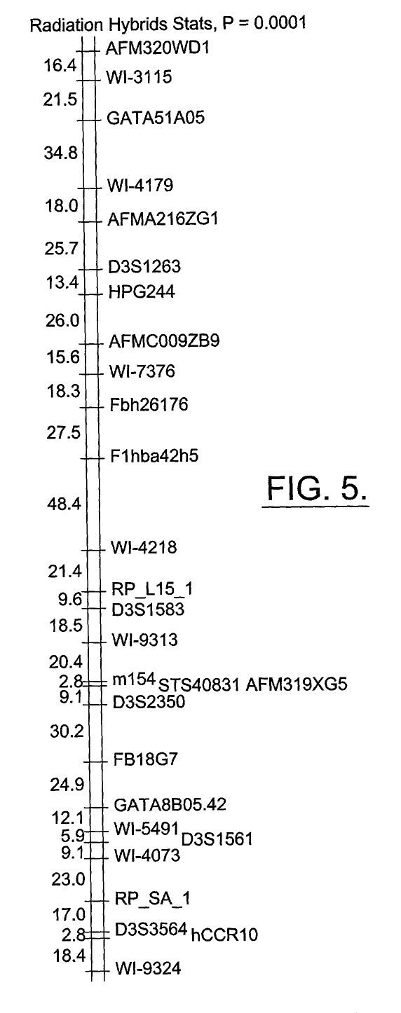

A search of the nucleotide and protein databases revealed that h26176 encodes a polypeptide that shares similarity with several calpain proteases, the greatest similarity being seen with the murine CAPN7 protein (EMB Accession Number AJ012475; SEQ ID NO 3). An alignment of the h26176 polypeptide with this murine protein is shown in FIG. 1. The alignment was generated using the Clustal method with PAM250 residue weight table and sequence identities were determined by pairwise alignment.

### EXAMPLE 2

### mRNA Expression of Clone h26176

Expression of the novel h26176 calpain protease was measured by TaqMan® quantitative PCR (Perkin Elmer Applied Biosystems) in cDNA prepared from the following human tissues: normal colon, colon carcinoma, normal liver, colon metastasis, normal lung, lung carcinoma, normal breast, and breast carinoma.

Probes were designed by PrimerExpress software (PE Biosystems) based on the h26176 sequence. The primers and probes for expression analysis of h26176 and β-2 microglobulin were as follows:

| | |
|---|---|
| h26176 Forward Primer | AATAGTATCGGATTGCTCCTTTGTG |
| h26176 Reverse Primer | GCCGGTAATTAACTTCTTATTAAAACG |
| h26176 TaqMan Probe | CATCACTGGCCATCAGTGCAGCTTATG |
| β-2 microglobulin Forward Primer | CACCCCCACTGAAAAAGATGA |
| β-2 microglobulin Reverse Primer | CTTAACTATCTTGGGCTGTGACAAAG |
| β-2 microglobulin TaqMan Probe | TATGCCTGCCGTGTGAACCACGTG |

The h26176 sequence probe was labeled using FAM (6-carboxyfluorescein), and the β2-microglobulin reference probe was labeled with a different fluorescent dye, VIC. The differential labeling of the target calpain protease sequence and internal reference gene thus enabled measurement in the same well. Forward and reverse primers and the probes for both β2-microglobulin and the target h26176 sequence were added to the TaqMano Universal PCR Master Mix (PE Applied Biosystems). Although the final concentration of primer and probe could vary, each was internally consistent within a given experiment. A typical experiment contained 200 nM of forward and reverse primers plus 100 nM probe for β-2 microglobulin and 600 nM forward and reverse primers plus 200 nM probe for the target h26176 sequence. TaqMan matrix experiments were carried out on an ABI PRISM 7700 Sequence Detection System (PE Applied Biosystems). The thermal cycler conditions were as follows: hold for 2 min at 50° C. and 10 min at 95° C., followed by two-step PCR for 40 cycles of 95° C. for 15 sec followed by 60° C. for 1 min.

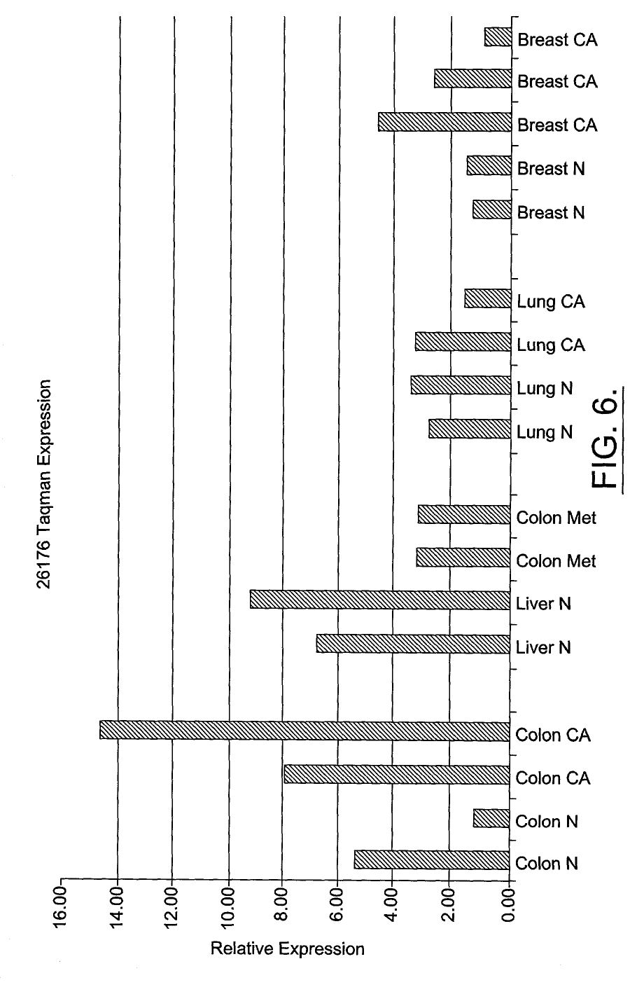

The following method was used to quantitatively calculate h26176 expression in the various tissues relative to β-2 microglobulin expression in the same tissue. The threshold cycle (Ct) value is defined as the cycle at which a statistically significant increase in fluorescence is detected. A lower Ct value is indicative of a higher mRNA concentration. The Ct value of the h26176 sequence is normalized by subtracting the Ct value of the β-2 microglobulin gene to obtain a _{Δ}Ct value using the following formula: _{Δ}Ct=Ctₕ₂₆₁₇₆−Ct_{β-2} microglobulin. Expression is then calibrated against a cDNA sample showing a comparatively low level of expression of the h26176 sequence. The ,Ct value for the calibrator sample is then subtracted from _{Δ}Ct for each tissue sample according to the following formula: _{ΔΔ}Ct=_{Δ}Ct₋ₛₐₘₚₗₑ−_{Δ}Ct−_{calbrator}. Relative expression is then calculated using the arithmetic formula given by 2^{-ΔΔCt}. Expression of the target h26176 sequence in each of the tissues tested was then graphically represented in FIG. 6.

The mRNA for the putative calpain protease h26176 is expressed in a variety of tumors. There was significant upregulation in colon carcinoma and breast carcinoma (FIG. 6). Accordingly, expression of the calpain protease is relevant to colon and breast carcinoma. In additional experiments, the gene was expressed in three out of four normal lung tissue samples but in 15 out of 16 lung carcinoma clinical samples (data not shown). Accordingly, expression of the calpain protease is relevant to lung carcinoma as well. This is consistent with the hypothesis that proteases may function in carcinogenesis by inactivating or activating regulators of cell cycle, differentiation, apoptosis, or other processes affecting cancer development and/or progression. In view of the fact that the gene is up-regulated in colon carcinoma, the gene is useful for inhibiting tumor progression. Inhibition of expression of this protease can thus be used to decrease the progression of carcinogenesis.

In addition, Northern blot experiments showed expression of the calpain protease in bone, ovary, T-cell, spleen, and kidney tissue. Accordingly, the protease is relevant to disorders involving these tissues.

In addition, expression has been observed in heart, neuronal tissue, monocytes, and prostate. Accordingly, expression of the gene is relevant to disorders involving these tissues.

Finally, expression has been observed in parathyroid tumor and in thymus. Accordingly, detection of expression or modulation of expression of the gene in these tissues, and particularly in disorders involving these tissues, is relevant.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Those skilled in the art will recognize, or be able to ascertain using no more than routine experimentation, many equivalents to the specific embodiments of the invention described herein. Such equivalents are intended to be encompassed by the following claims.

That which is claimed:

1. An isolated nucleic acid molecule comprising a nucleotide sequence selected from the group consisting of:
(a) a nucleotide sequence encoding a polypeptide comprising the amino acid sequence set forth in SEQ ID NO:2;
(b) a nucleotide sequence encoding the polypeptide encoded by the cDNA insert of the plasmid deposited with ATCC as Patent Deposit Number PTA-1649; and
(c) a nucleotide sequence complementary to the nucleotide sequence of (a) or (b).

2. The nucleic acid molecule of claim 1, wherein said nucleic acid molecule comprises a nucleotide sequence selected from the group consisting of:
(a) the nucleotide sequence set forth in SEQ ID NO:1;
(b) the nucleotide sequence of the cDNA insert of the plasmid deposited with ATCC as Patent Deposit Number PTA-1649; and
(c) a nucleotide sequence complementary to the nucleotide sequence of (a) or (b).

3. The nucleic acid molecule of claim 1 further comprising vector nucleotide sequences.

4. The nucleic acid molecule of claim 1 further comprising nucleotide sequences encoding a heterologous polypeptide.

5. A host cell containing the nucleic acid molecule of claim 3.

6. The host cell of claim 5, wherein said host cell is a mammalian host cell.

7. A nonhuman mammalian host cell containing the nucleic acid molecule of claim 1.

8. An isolated nucleic acid molecule comprising a nucleotide sequence selected from the group consisting of:
(a) a nucleotide sequence encoding a sequence variant of the amino acid sequence set forth in SEQ ID NO:2 wherein the nucleotide sequence has at least about 75% sequence identity to the nucleotide sequence set forth in SEQ ID NO:1 and the sequence variant has calpain protease activity;
(b) a nucleotide sequence encoding a sequence variant of the amino acid sequence encoded by the cDNA insert of the plasmid deposited with ATCC as Patent Deposit Number PTA-1649, wherein said nucleotide sequence has at least about 75% sequence identity to the nucleotide sequence of the cDNA insert of the plasmid deposited with ATCC as Patent Deposit Number PTA-1649 and the sequence variant has calpain protease activity; and
(c) a nucleotide sequence complementary to the nucleotide sequence of (a) or (b).

9. The nucleic acid molecule of claim 8, wherein said nucleic acid molecule comprises a nucleotide sequence selected from the group consisting of:
(a) a nucleotide sequence encoding a sequence variant of the amino acid sequence set forth in SEQ ID NO:2, wherein said nucleotide sequence has at least about 85% sequence identity to the nucleotide sequence set forth in SEQ ID NO:1 and said sequence variant has calpain protease activity;
(b) a nucleotide sequence encoding a sequence variant of the polypeptide encoded by the cDNA insert of the plasmid deposited with ATCC as Patent Deposit Number PTA-1649, wherein said nucleotide sequence has at least about 85% sequence identity to the nucleotide sequence of the cDNA insert of the plasmid deposited with ATCC as Patent Deposit No. PTA-1649; and
(c) a nucleotide sequence complementary to (a) or (b).

10. The isolated nucleic acid molecule of claim 9, wherein said nucleotide sequence is selected from the group consisting of:
(a) a nucleotide sequence encoding a sequence variant of the amino acid sequence set forth in SEQ ID NO:2, wherein said nucleotide sequence has at least about 95% sequence identity to the nucleotide sequence set forth in SEQ ID NO:1 and said sequence variant has calpain protease activity;
(b) a nucleotide sequence encoding a sequence variant of the polypeptide encoded by the cDNA insert of the plasmid deposited with ATCC as Patent Deposit Number PTA-1649, wherein said nucleotide sequence has at least about 95% sequence identity to the nucleotide sequence of the cDNA insert of the plasmid deposited with ATCC as Patent Deposit No. PTA-1649; and
(c) a nucleotide sequence complementary to the nucleotide sequence of (a) or (b).

11. The isolated nucleic acid molecule of claim 10, wherein said nucleotide sequence is selected from the group consisting of:
(a) a nucleotide sequence encoding a sequence variant of the amino acid sequence set forth in SEQ ID NO:2, wherein said nucleotide sequence has at least 98% sequence identity to the nucleotide sequence set forth in SEQ ID NO:1 and said sequence variant has calpain protease activity;
(b) a nucleotide sequence encoding a sequence variant of the polypeptide encoded by the cDNA insert of the plasmid deposited with ATCC as Patent Deposit Number PTA-1649, wherein said nucleotide sequence has at least 98% sequence identity to the nucleotide sequence of the cDNA insert of the plasmid deposited with ATCC as Patent Deposit No. PTA-1649; and
(c) a nucleotide sequence complementary to the nucleotide sequence of (a) or (b).

12. An isolated nucleic acid molecule comprising a nucleotide sequence selected from the group consisting of:
(a) a nucleotide sequence consisting of at least 20 contiguous nucleotides of the nucleotide sequence set forth in SEQ ID NO:1;
(b) a nucleotide sequence consisting of at least 20 contiguous nucleotides of the nucleotide sequence of the cDNA insert of the plasmid deposited with ATCC as Patent Deposit Number PTA-1649;
(c) a nucleotide sequence encoding a fragment of the amino acid sequence set forth in SEQ ID NO:2, wherein said fragment consists of at least about 15 contiguous amino acids of the amino acid sequence set forth in SEQ ID NO:2 and has calpain protease activity;
(d) a nucleotide sequence encoding a fragment of the amino acid sequence encoded by the cDNA insert of the plasmid deposited with ATCC as Patent Deposit Number PTA-1649, wherein the fragment comprises at least 15 contiguous amino acids of the amino acid sequence encoded by the cDNA insert of the plasmid deposited with ATCC as Patent Deposit Number PTA-1659 and has calpain protease activity; and
(e) a nucleotide sequence complementary to the nucleotide sequence of (a), (b), (c), or (d).

13. An isolated polypeptide comprising an amino acid sequence selected from the group consisting of:
(a) the amino acid sequence of a fragment of the amino acid sequence shown in SEQ ID NO:2, wherein said fragment comprises at least 15 contiguous amino acids of the amino acid sequence shown in SEQ ID NO:2 and has calpain protease activity; or
(b) the amino acid sequence of a fragment of the amino acid sequence encoded by the cDNA insert of the plasmid deposited with ATCC as Patent Deposit Number PTA-1649, wherein the fragment has calpain protease activity and comprises at least 15 contiguous amino acids of the amino acid sequence encoded by the cDNA insert of the plasmid deposited with ATCC as Patent Deposit Number PTA-1649;
(c) the amino acid sequence of a sequence variant of the amino acid sequence set forth in SEQ ID NO:2 wherein said sequence variant has calpain protease activity and is encoded by a nucleic acid molecule that hybridizes to the complement of the nucleotide sequence shown in SEQ ID NO:1 under stringent conditions;
(d) the amino acid sequence of a sequence variant of the amino acid sequence encoded by the cDNA insert of the plasmid deposited with ATCC as Patent Deposit Number PTA-1649, wherein the sequence variant has calpain protease activity and is encoded by a nucleic acid molecule which hybridizes to the complement of the nucleotide sequence set forth in SEQ ID NO: lunder stringent conditions; and
(e) a polypeptide which is encoded by a nucleic acid molecule comprising a nucleotide sequence which is at least 65% identical to a nucleic acid comprising the nucleotide sequence of SEQ ID NO:1, or a complement thereof.

14. The isolated polypeptide of claim 13, wherein said polypeptide comprises an amino acid sequence selected from the group consisting of:
(a) the amino acid sequence set forth in SEQ ID NO:2; and
(b) the amino acid sequence encoded by the cDNA insert of the plasmid deposited with ATCC as Patent Deposit Number PTA-1649.

15. The polypeptide of claim 13 further comprising heterologous amino acid sequences.

16. An antibody which selectively binds to a polypeptide of claim 13.

17. A method for producing a polypeptide of claim 13, said method comprising culturing the host cell containing a nucleic acid molecule encoding said polypeptide under conditions in which the nucleic acid molecule is expressed.

18. The method of claim 17 wherein said polypeptide is selected from the group consisting of:
(a) the amino acid sequence set forth in SEQ ID NO:2; and
(b) the amino acid sequence encoded by the cDNA insert of the plasmid deposited with ATCC as Patent Deposit Number PTA-1649.

19. A method for detecting the presence of a polypeptide of claim 13 in a sample, said method comprising:
(a) contacting the sample with a compound that selectively binds to a polypeptide of claim 13; and
(b) determining whether the compound binds to the polypeptide in the sample.

20. The method of claim 19, wherein the compound which binds to the polypeptide is an antibody.

21. A kit comprising a compound which selectively binds to a polypeptide of claim 13 and instructions for use.

22. A method for detecting the presence of a nucleic acid molecule of claim 1 in a sample, comprising the steps of:
(a) contacting the sample with a nucleic acid probe or primer which selectively hybridizes to the nucleic acid molecule; and
(b) determining whether the nucleic acid probe or primer binds to a nucleic acid molecule in the sample.

23. The method of claim 22, wherein the sample comprises mRNA molecules and is contacted with a nucleic acid probe.

24. A kit comprising a compound which selectively hybridizes to a nucleic acid molecule of claim 1 and instructions for use.

25. A method for identifying a compound which binds to a polypeptide of claim 13 comprising the steps of:
(a) contacting a polypeptide or claim 13, or a cell expressing a polypeptide of claim 13 with a test compound; and
(b) determining whether the polypeptide binds to the test compound.

26. The method of claim 25, wherein the binding of the test compound to the polypeptide is detected by a method selected from the group consisting of:
(a) detection of binding by direct detecting of test compound/polypeptide binding;
(b) detection of binding using a competition binding assay;
(c) detection of binding using an assay for calpain protease activity.

27. A method for modulating the activity of a polypeptide of claim 13 comprising contacting a polypeptide or a cell expressing a polypeptide of claim 13 with a compound which binds to the polypeptide in a sufficient concentration to modulate the activity of the polypeptide.

28. A method for identifying a compound which modulates the activity of a polypeptide of claim 13, comprising:
(a) contacting a polypeptide of claim 13 with a test compound; and
(b) determining the effect of the test compound on the activity of the polypeptide to thereby identify a compound which modulates the activity of the polypeptide.
